(19) United States
(12) Patent Application Publication
(10) Pub. No.: US 2026/0014314 A1
(43) Pub. Date: Jan. 15, 2026

(54) MULTIMODAL THERAPY DELIVERY SYSTEM

(71) Applicant: University of Iowa Research Foundation, Iowa City, IA (US)

(72) Inventors: Sandeep Laroia, Iowa City, IA (US); Fatima Toor, Coralville, IA (US)

(73) Assignee: University of Iowa Research Foundation, Iowa City, IA (US)

(21) Appl. No.: 19/266,664

(22) Filed: Jul. 11, 2025

Related U.S. Application Data
(60) Provisional application No. 63/669,850, filed on Jul. 11, 2024.

(51) Int. Cl. A61M 5/158; A61B 18/00; A61B 18/14; A61B 18/20; A61N 5/06; A61N 5/067

(52) U.S. Cl. CPC A61M 5/158; A61B 18/14; A61N 5/0601; A61N 5/067; A61B 2018/00577; A61B 2018/2005; A61B 2018/206; A61M 2205/052; A61N 2005/0612; A61N 2005/0626; A61N 2005/063; A61N 2005/0659

(57) ABSTRACT

A multimodal therapy delivery system configured to deliver both biochemical material and energy therapies, the multimodal therapy delivery system includes at least one mid-infrared (MIR) laser, at least one MIR transparent fluoride fiber coupled to each of the at least one MIR laser, and a needle assembly with the at least one MIR transparent fluoride fiber threaded therethrough. The multimodal therapy delivery system is configured to deliver biochemical material through the needle assembly and wherein the multimodal therapy delivery system is further configured to deliver energy from the at least one MIR laser through the at least one MIR transparent fluoride fiber threaded through the needle assembly.

### RELATED APPLICATIONS

This application claims priority to U.S. Provisional Patent Application No. 63/669,850, filed Jul. 11, 2024, entitled “Multimodal Therapy Delivery System,” hereby incorporated by reference in its entirety.

### FIELD OF THE INVENTION

The present invention relates generally, but not exclusively, to a multimodal therapy delivery system and components thereof including mid-infrared laser delivery systems.

### BACKGROUND

The present disclosure relates to and addresses a number of different problems which are seemingly unrelated without having the benefit of this disclosure. One set of problems relates generally to locoregional therapies where therapeutic agents such as drugs or biochemicals are used. Locoregional therapies means that the therapeutic agent is delivered in close vicinity of the disease by the way of blood stream or directly into the lesion. The direct injection into the tumor or pathology is called intralesional therapy.

Intralesional therapy includes target therapies, especially the ones directed towards foci of cancers. Generally, intralesional therapies are successful because they provide change in the microenvironment of the tumor, which is often not possible by systemic therapy, they have relatively minor side effects compared to systemic therapies, often limited to injection site reactions, and provide the ability to target early stage cancer with more potent therapies.

Various modalities can be used to target focal lesions. These modalities may be classified broadly as liquid delivering and energy delivering therapies. Liquid based therapies include, without limitation, radiopharmaceuticals, immune modulators, antibodies, cytokines or specifically targeted molecules, and chemical ablation therapies like alcohol or acetic acid etc. Even traditional chemotherapy agents like Cisplatin and Vinblastine may be used. Energy delivering therapies include, without limitation, radiofrequency (RF), microwave (MW), electric energy (EE), and cold based therapies (cryo).

However though great advancements have been made in specifically targeted therapies, currently available devices lack the sophistication required to tailor this therapy for specific clinical situations.

Off the shelf generic needles are used in local intralesional/intratumoral access to introduce a drug into a lesion. Typical straight needles have a single end hole and so will typically inject only around that focal point. Similarly an energy probe delivered through such a linear fashion will be limited to tissues which are available to it in a straight line.

The operator can manipulate the needle or do multiple injections to follow different trajectories. This may lead to undesired side effects like more pain, more bleeding, risk of tumor spread around needle entrance or many a times simply not feasible because of limited access due to other vital structures present in close vicinity. Also, many a times the drug might be toxic to the surrounding healthy tissue, so proper control over the injected volume is needed, so that it targets the diseased tissue only.

Placing a needle using image guidance into a target lesion is often straightforward, ensuring that the injected therapy is distributed throughout the target lesion without leakage into the surrounding tissue is not. Thus, there are numerous problems with the use of needles to deliver therapeutic agents to target lesions.

Another set of problems relates to the use of traditional lasers in applications including medical applications typically operating in the UV (λ₀=250-350 nm) and NIR (λ₀=800-1100 nm) wavelengths. Although lasers would appear to be useful for tumor ablation, there are numerous problems in doing so. For example, existing UV, NIR laser technologies are bulky light sources which may be housed in separate rooms or in large containments for safety reasons. This further exacerbates issues with bringing light from MIR lasers to a surgical bed using optical fibers as the fibers experience bending and transmission losses which may limit freedom of motion necessary. Thus, there are issues with the use of MIR generally.

Lasers operating in the mid-infrared (MIR) waveband (λ₀=3-13 μm) can overcome many of these problems. MIR lasers can also selectively ablate tumor tissue with respect to healthy tissue, enabling less invasive surgical procedures. Quantum cascade lasers (QCLs) which operate at room temperature and are power efficient. However in particular, the use of surgical ablation using QCLs has not been studied in depth to determine safety and efficacy.

What is needed are new and innovative technologies which enhance therapy delivery and/or provide as system for multimodal therapy delivery.

### SUMMARY

Therefore, it is a primary object, feature, or advantage to improve over the state of the art.

It is a further object, feature, or advantage to provide a multimodal therapy delivery system which provides for combined laser and immunotherapy.

Another object, feature, or advantage is to provide quantum-engineered, mid-infrared (MIR) (3-13 μm wavelength emission) lasers for use in laser thermal ablation.

Yet another object, feature, or advantage to deliver MIR laser energy to tissue in a manner which reduces losses.

A still further object, feature, or advantage is to provide a multi-prong needle which allows for simultaneous delivery of laser energy and/or biochemical liquids to tissue.

Another object, feature, or advantage is to provide methods, system, and apparatuses for volumetrically ablating tissue.

Yet another object, feature, or advantage is to provide multimodal therapy delivery in a manner which is tunable.

One or more of these and/or other objects, features, or advantages will become apparent from the specification and claims that follow. No single embodiment need provide each and every object, feature, or advantage. Instead, different embodiments may have different objects, features, or advantages.

According to one aspect, a multimodal therapy delivery system configured to deliver both biochemical material and energy therapies, the multimodal therapy delivery system is provided. The system includes at least one mid-infrared (MIR) laser, at least one MIR transparent fluoride fiber coupled to each of the at least one MIR laser, and a needle assembly, with the at least one MIR transparent fluoride fiber threaded therethrough. The multimodal therapy delivery system is configured to deliver biochemical material through the needle assembly and wherein the multimodal therapy delivery system is further configured to deliver energy from the at least one MIR laser through the at least one MIR transparent fluoride fiber threaded through the needle assembly. The needle assembly may include a multi-prong needle for delivering the biochemical material. The needle assembly may include a plurality of side holes positioned in a spiral fashion along a length of a needle within the needle assembly. The needle assembly may include at least one curved channel the needle assembly may further include a side hole. The needle assembly may further include an outer sheath. The multimodal therapy delivery system may further include a delivery mechanism configured to control flow of the biochemical material and delivery of light from the MIR laser. The delivery mechanism may include a pump configured to separately delivery at least one biochemical material to a plurality of different channels of the needle assembly. The needle assembly may include a metallic structure such as stainless steel having a plurality of channels. Each of the at least one MIR transparent fiber is textured for diffused light output. The multimodal therapy delivery system may further include a control system for operating the multimodal therapy delivery system wherein the control system provides for controlling amount of energy delivered from the at least one MIR laser to a target based on position of the at least one MIR transparent fluoride fiber and calculated losses associated with delivery of energy from the at least on MIR laser to the target. The at least one MIR transparent fluoride fiber may include at least one of indium fluoride or zirconium fluoride. The multimodal therapy delivery system may further include at least one delivery mechanism operatively connected to one or more reservoirs of biochemical material, a control system operatively connected to the at least one delivery mechanism and the at least one MIR laser, and a display operatively connected to the control system.

According to another aspect, a method includes using the multimodal therapy delivery system to volumetrically ablate tissue. The method may further include positioning the multimodal therapy delivery system by bending the at least one MIR transparent fluoride fiber prior to delivery of the energy from the at least one MIR laser.

According to another aspect, a therapy delivery system includes at least one mid-infrared (MIR) laser, a plurality of MIR transparent fibers coupled to one of the at least one MIR laser, and a coupling for each of the at least one of the plurality of MIR transparent fibers, the coupling comprising a housing assembly and a plurality of lenses. Each of the plurality of MIR transparent fibers may be textured. Each of the at least one MIR transparent fibers may comprise fluoride.

According to another aspect, a needle assembly for use in a multimodal therapy delivery system includes a conduit needle having an outer sheath, a plurality of channels in the conduit needle, an exit hole from each of the plurality of channels, at least one fluid injecting delivery needle for delivering a therapeutic fluid, at least one energy delivery needle for delivering laser energy, and at least one laser fiber operatively connected to a corresponding one of the at least one energy delivery needle for threading through a corresponding one of the plurality of channels to deliver the laser energy. The needle assembly is configured for delivering the therapeutic fluid and the laser energy simultaneously.

According to another aspect, a method for delivery of multimodal therapy to a patient is provided. The method includes delivering at least one biochemical through a first channel of a needle assembly to tissue of the patient and simultaneously delivering energy from a mid-infrared (MIR) laser to the tissue of the patient through a MIR transparent fiber positioned within a second channel of the needle assembly.

According to another aspect, a method of volumetrically ablating a volume of tissue is provided. The method includes providing a multimodal therapy delivery system configured to deliver both biochemical material and energy therapies, the multimodal therapy delivery system including: (1) at least one mid-infrared (MIR) laser having a wavelength within a range of 3 to 13 μm; (2) at least one MIR transparent fluoride fiber coupled to each of the at least one MIR laser using a coupling; (3) a needle assembly, with the at least one MIR transparent fluoride fiber threaded therethrough; and (4) wherein the multimodal therapy delivery system is configured to deliver biochemical material through the needle assembly and wherein the multimodal therapy delivery system is further configured to deliver energy from the at least one MIR laser through the at least one MIR transparent fluoride fiber threaded through the needle assembly. The method further includes laser ablating the volume of tissue using the multimodal therapy delivery system. A wavelength of the at least one MIR laser is in a range of 3 microns to 13 microns. The volume of the tissue being volumetrically ablated is in a range of 5-100 mm³ and wherein the multimodal therapy system is configured to determine parameters at least based in part on the volume of the tissue being volumetrically ablated. The coupling may include a housing assembly and a plurality of lenses and wherein the coupling provides for at least 90 percent coupling.

### BRIEF DESCRIPTION OF THE FIGURES

FIG. 1A is a block diagram illustrating one example of a system for multimodal therapy.

FIG. 1B is a pictorial representation of one example of a system for multimodal therapy.

FIG. 2 further illustrates a needle assembly configured for multimodal therapy.

FIG. 3 further illustrates the needle assembly.

FIG. 4 further illustrates the needle assembly.

FIG. 5 further illustrates the needle assembly with its rail system.

FIG. 6 illustrates the needle assembly with staggered openings therein.

FIG. 7 further illustrates a delivery system.

FIG. 8 illustrates absorption for different wavelengths.

FIG. 9 illustrates microscopic images of ablated chicken breast.

FIG. 10 illustrates coagulated and charred diameters and depths over time for Study T.

FIG. 11 illustrates the coagulated and charred diameters and depths for various frequencies for Study F.

FIG. 12 illustrates the coagulated and charred diameters and depths for various duty cycles for Study D.

FIG. 13 summarizes validation of experimental results for an ablation simulation. (A) The surface temperature (° C.) of the tissue after 120-sec of laser exposure. The max and min temperatures are 164° C. and 27.7° C., respectively. (B) The coagulative necrosis profile after 120-sec. The diameter and depth are 2.09-mm and 0.74-mm, respectively. (C) The ray trajectories and penetration into the tissue. Rays with the max initial power of 53-μW penetrate 0.15-mm and are reduced to 0.53-μW. (D&E) Coagulated Depth and Diameter growth over time. Experimentally measured and COMSOL simulated values are compared.

FIG. 14 illustrates thermographic validation. (A) thermographic image of the hotspot generated during chicken breast ablation after 120-sec at 0.2 W. The maximum reported temperature varies from 100° C. to 120° C. (B) the temperature profile of the tissue surface as calculated by COMSOL. The inset is the size of the iris. (C) thermographic image of the hotplate set to either 185° C. or 100° C. with/out the iris.

FIG. 15 illustrates the center of a textured fiber length.

FIG. 16 illustrates an end of the textured fiber length.

FIG. 17 illustrates a quantum cascade laser in a horizontal high heat load (HHLH) package.

FIG. 18 further illustrates the fully assembled coupler.

FIG. 19 illustrates at panel (A), tracks configured such that the optical fiber maintains different bend radii; (B) fiber wound around a spool from the laser to the detector; (C) fiber wound around the spool; (D) a detector connected to a computer to display the detected voltage signal.

FIG. 20 illustrates results from the experimental testing for different types of fiber (InF3 and ZrF4) of different sizes where the bend radii was defined by the tracks.

FIG. 21 illustrates results for different types of fibers and sizes.

FIG. 22 illustrates representative geometry, meshing, and ray trajectories for a computation model.

FIGS. 23A, 23B, 23C, 23D, 23E, 23F, 23G, 23H illustrate results of the computational simulations for different angles, for different types of fibers, of different sizes.

FIG. 24 illustrates an experimental setup which includes laser components and a programmable syringe pump.

FIG. 25 is a figure illustrating results obtained using the experimental setup. The time (minutes) and temperature (degrees Celsius) are shown as well as the ablation zone diameter.

FIG. 26 illustrates an example of the needle assembly.

FIG. 27A is a photograph showing chicken breast tissue after fiber (untextured) ablation, FIG. 27B is a photograph showing the tissue, FIG. 27C is a graph showing temperature at different times using a 1000 mW/560 mW laser, no water bath. FIG. 27D illustrates that for untextured fiber, the mid-IR light comes out from the fiber end as confirmed by the tissue ablation.

FIG. 28A is a photograph showing chicken breast tissue after fiber (textured) ablation with 1000 mW (560 mW) for 3 minutes, not water bath, placed in room temperature for 2 hours with the initial temperature 18.3 degrees. FIG. 28B is a further photograph showing the chicken breast. FIG. 28C is a graph showing temperatures at different times. The fiber was burnt in 25 seconds. FIG. 28D illustrates that with the textured fiber, the mid-IR light comes out longitudinally across the textured fiber length as confirm with the tissue ablation.

FIG. 29 illustrates one example for a laser interstitial thermal therapy probe.

### DETAILED DESCRIPTION

The present disclosure describes a multimodal therapy delivery system. The multimodal therapy delivery system may provide for both lasers which are configured to provide for laser ablation and delivery of therapeutic agents such as biochemical liquids. This multimodal therapy may be used to target tumors or pathology.

The multimodal therapy delivery system provides for locoregional therapies. Locoregional therapies means that the therapeutic agent is delivered in close vicinity of the disease by the way of blood stream or directly into the lesion. The direct injection into the tumor or pathology is called intralesional therapy.

Intralesional therapy includes target therapies, especially the ones directed towards foci of cancers. Generally, intralesional therapies are successful because they provide change in the microenvironment of the tumor, which is often not possible by systemic therapy, they have relatively minor side effects compared to systemic therapies, often limited to injection site reactions, and provide the ability to target early stage cancer with more potent therapies.

Various modalities can be used to target focal lesions. These modalities may be classified broadly as liquid and energy delivering fibers. Liquid based therapies include, without limitation, radiopharmaceuticals, immune modulators, antibodies, cytokines or specifically targeted molecules, and chemical ablation therapies like alcohol or acetic acid etc. Even traditional chemotherapy agents like Cisplatin and Vinblastine may be used. Energy delivering fibers include, without limitation, radiofrequency (RF), microwave (MW), electric energy (EE), and cold based therapies (cryo).

However though great advancements have been made in specifically targeted therapies, currently available devices lack the sophistication required to tailor this therapy for specific clinical situations.

Off the shelf generic needles are used in local intralesional/intratumoral access to introduce a drug into a lesion. Typical straight needles have a single end hole and so will typically inject only around that focal point. Similarly energy probe delivered through such a linear fashion will be limited to tissues which are available to it in a straight line.

The operator can manipulate the needle or do multiple injections to follow different trajectories. This may lead to undesired side effects like more pain, more bleeding, risk of tumor spread around needle entrance or many a times simply not feasible because of limited access due to other vital structures present in close vicinity. Also, many a times the drug might be toxic to the surrounding healthy tissue, so proper control over the injected volume is needed, so that it targets the diseased tissue only.

Placing a needle using image guidance into a target lesion assists in ensuring that the injected therapy is distributed throughout the target lesion without leakage into the surrounding tissue is not. Using image guidance such as ultrasound guidance enhances accuracy relative to palpation-guided methods. Arias-Buría, J. L., Borrella-Andrés, S., Rodríguez-Sanz, J., López-de-Celis, C., Malo-Urriés, M., Fernández-de-las-Peñas, C., Gallego-Sendarrubias, G. M., González-Rueda, V., Pérez-Bellmunt, A., & Albarova-Corral, I. (2023). Precision and Safety of Ultrasound-Guided versus Palpation-Guided Needle Placement on the Patellar Tendon: A Cadaveric Study. Life, 13(10), 2060. For example with image guidance such as ultrasound guidance, the tip of the needle may be considered to be accurately placed if it is within a small threshold distance of the target, the threshold distance may be 3 mm, 2 mm, 1 mm, 0.5 mm, 0.25 mm or less. Use of image guidance allows for consistent needle placement within the threshold distance. In addition, precise placement allows for a larger surface of contact with the target issue and better distribution throughout the target lesion. Thus, use of image guidance allows for precise needle placement, reduces risk of damaging surrounding structures and enhances efficacy of the treatment relative to less precise placement.

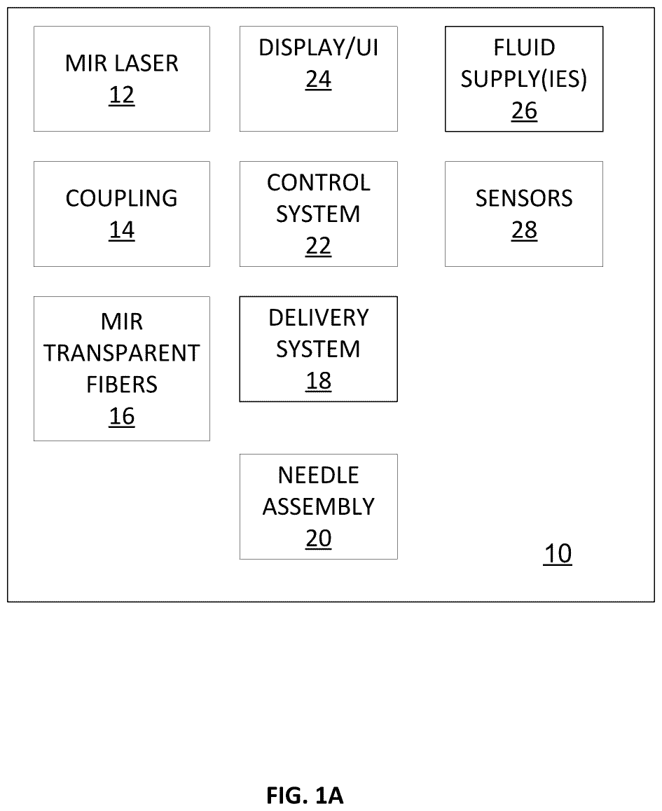

FIG. 1A is a block diagram illustrating one example of a multimodal therapy delivery system 10. The multimodal therapy delivery system 10 may include at least one laser such as a mid-infrared laser 12 as well as one or more fluids which contains biochemicals or therapeutic agents from one or more fluid supplies 26.

Light or energy from the MIR laser 12 may travel through a coupling 14. The coupling 14 preferably is optimized to limit loss. The coupling 14 connects the MIR laser 12 to one or more MIR transparent fibers 16. A delivery mechanism 18 provides for delivering light from one or more of the MIR transparent fibers 16 to the needle assembly 20 as well as delivering one or more fluids from the fluid supplies 26 to the needle assembly 20.

A control system 22 is shown which may be used to perform any number of control operations and may be configured to receive sensor measurements from any number of different sensors including sensors 28 which may be operatively connected to the control system 22. The control system 22 may also be operatively connected to the on more MIR lasers 12 in order control output. A display 24 which may include a user interface may be operatively connected to the control system 12. The display may display information about operations performed with the multimodal therapy delivery system 10, its settings, or other information. The display may be a touch display which allows a user to change settings or otherwise configuration operation of the multimodal therapy delivery system 10.

Although FIG. 1A provides an overview of a system is to be understood that more or fewer components may be used in a particular embodiment of the system.

FIG. 1B is a pictorial representation illustrating a system 10 configured to deliver both laser light and biochemicals to lesions, simultaneously, and in a customizable manner. As shown in FIG. 1B, the energy delivery component will be based on small formfactor, quantum-engineered, mid-infrared (MIR) lasers coupled to fibers that can be wavelength- and power-tuned for precise tissue ablation. The fibers when positioned within a body may bend. Note also that in FIG. 1B a probe such as an ultrasound probe is shown which may be used for generating imagery to assist in guiding a needle assembly and its individuals needles for delivery of laser energy through MIR laser coupled fibers and/or biochemicals.

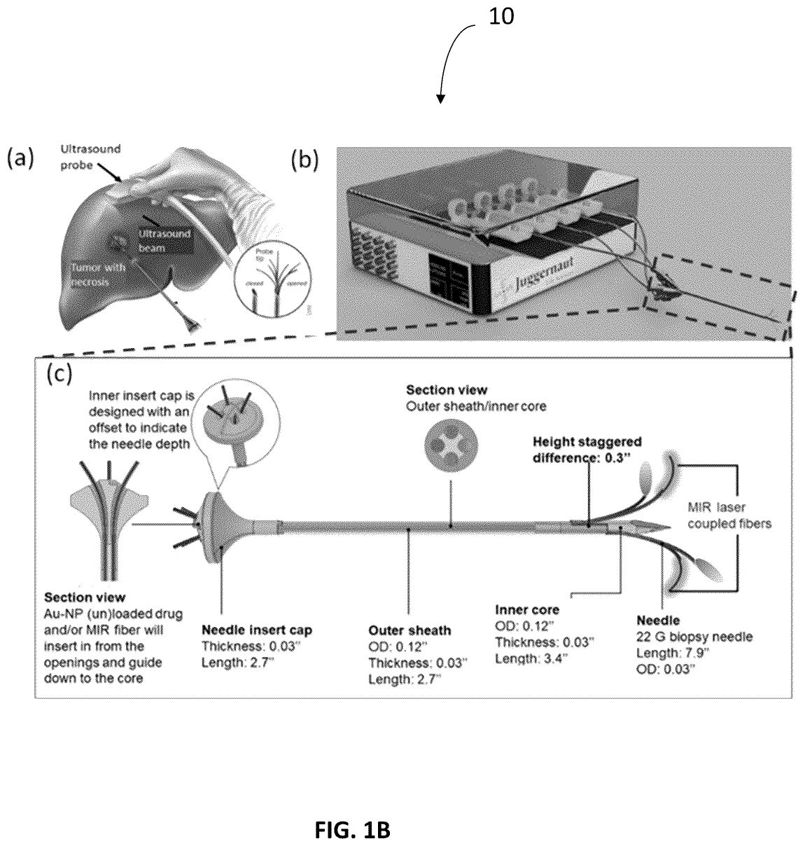

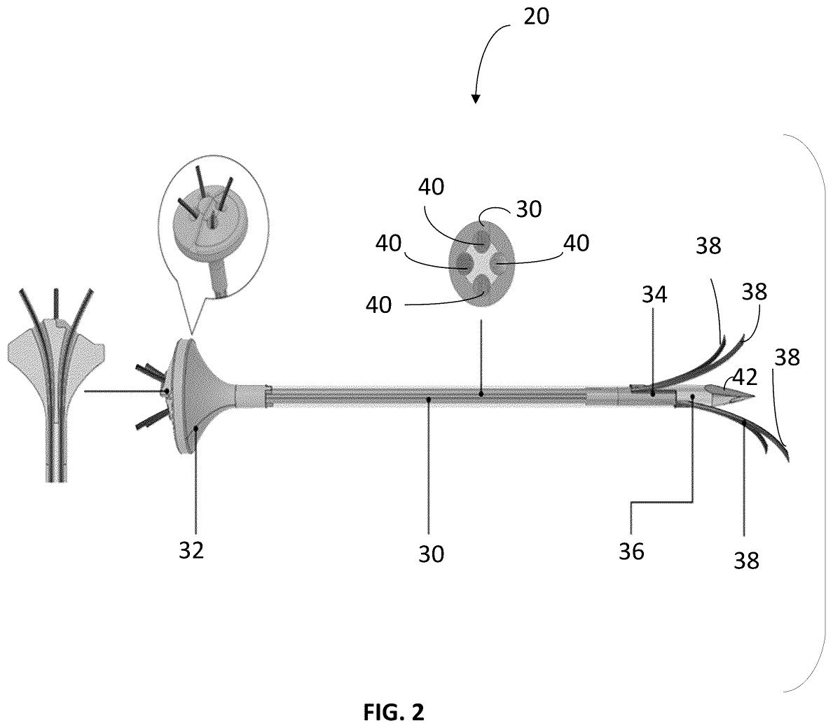

FIG. 2 illustrates one example of a needle assembly 20 with a multi-prong design. The needle assembly may be formed generally of a metallic structure such as stainless steel. As shown in FIG. 2, the needle assembly 20 includes an outer sheath 30 which has an inner core 36. The inner core 36 includes a plurality of channels 40. Each of these channels 40 may provide for receiving a different needle which may be a fluid injecting needle or an energy delivery needle. In some embodiments, one or more of the channels may be a curved channel. The benefit of a curved channel is that it allows for potentially greater or more effective coverage of a tumor as the tumor may be approached from a different angle or position. A needle insertion cap 32 is shown at one end of the needle assembly 20. Needles 38 may be inserted in through openings in the needle insertion cap 32 and be guided down through the needle insertion cap 32 and through a corresponding channel 40 of the inner core 36. Each of the needles 38 may then extend outwardly on an opposite end of the needle assembly 20 from the needle insertion cap 32.

The outer sheath 30 may terminate in an anchor needle tip 42. The anchor needle tip 42 also may include a lumen and multiple holes near the tip end and thus can also be used for volumetric injections. It should be understood that there may be differing numbers of channels or lumens present in the anchor needle. For example, there may be a single channel, a double channel, a triple channel, a quadruple channel or more.

In some embodiments, a single side hole may be present to guide a single therapy needle. The use of a side hole allows the needle to be angled more steeply in order to reach tissue which is normally unreachable.

The needle assembly 20 may include a hole and rail arrangement of the exit holes for injecting needles or treatment needles 38. Such an arrangement assists in directing an injecting needle 38 to its desired course. The injecting needles 38 may include side holes placed in a spiral arrangement and configured to infuse therapy material in a uniform manner. Each needle 38 may be positioned in a staggered manner in order to provide an operator greater control over the volume of tissue being covered. Each needle 38 may be placed and removed independently from other needles. Each needle 38 may have its own hub so combination therapies or variable amounts of drugs can be injected through each of the needles 38. The needles 38 are configured such that other therapy modalities including laser fibers may be introduced through the needles 38 to allow for multi-modal therapy.

In some embodiments, the needle assembly 20 is advantageous in that when distributing liquids, a significant amount of volumetric coverage may be provided. The volumetric coverage may provide for a spherical deposition of liquids.

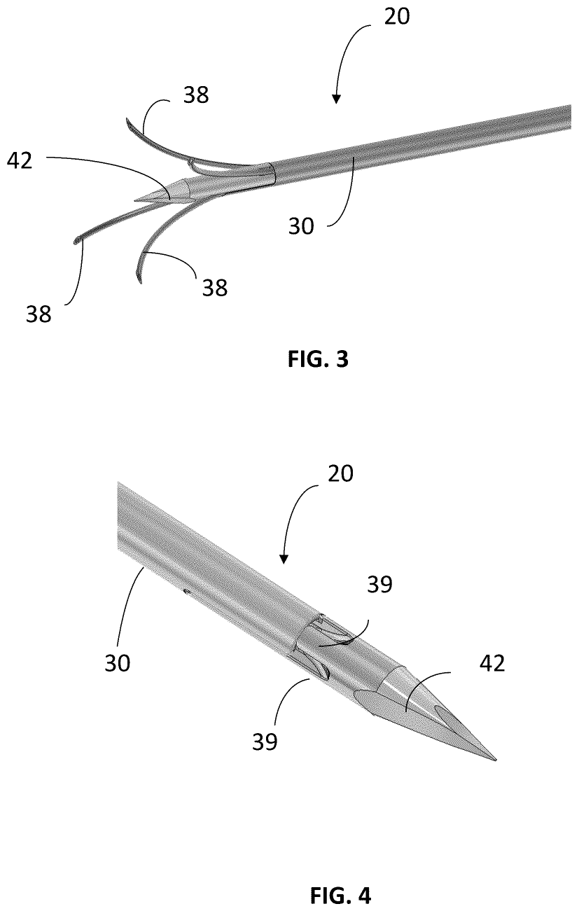

FIG. 4 further illustrates a portion of the needle assembly 20 with its outer sheath 30 or anchor needle with anchor needle tip 42. A plurality of exit holes 39 are shown.

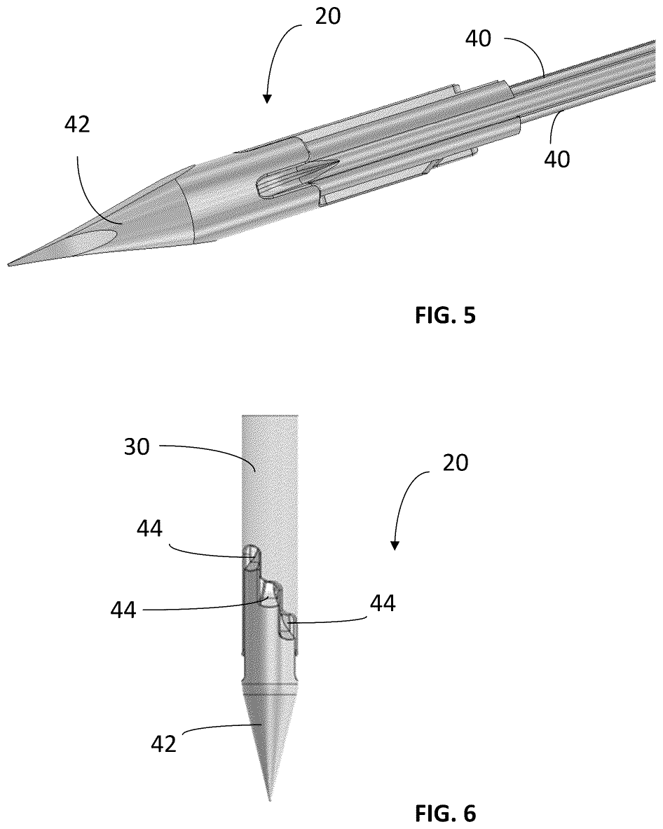

FIG. 5 further illustrates a portion of the needle assembly 20 with the plurality of channels 40 in a hole and rail arrangement.

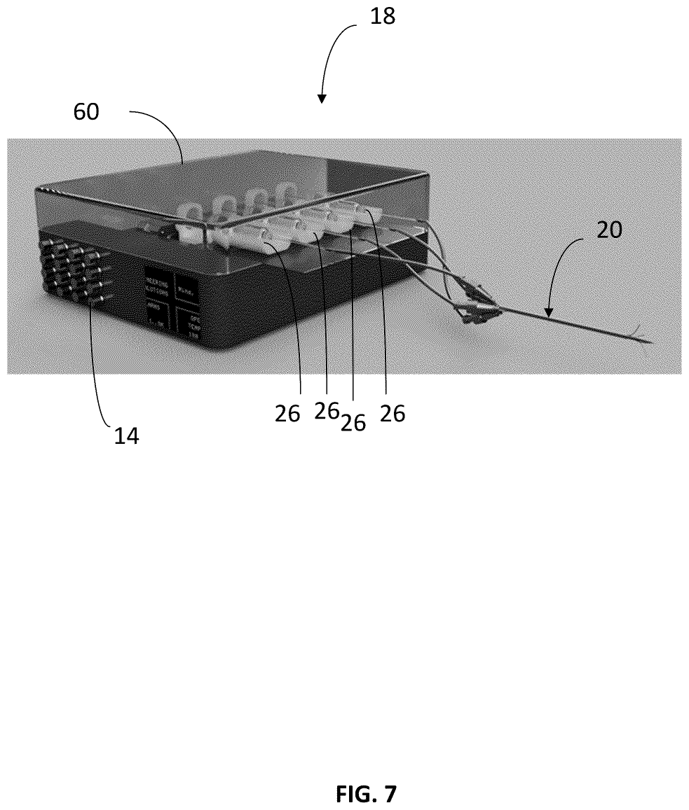

FIG. 6 further illustrates a portion of the needle assembly 20 with a plurality of side holes 44 positioned in a spiral fashion along a length of the needle assembly.

It should be understood that the needle assembly provides numerous advantages including avoiding the need to perform multiple injections. This is a significant advantage because if multiple injections are performed this may not be safe as it may lead to seeding of the tumor along the tract. Another significant advantage is reliable coverage of an entire volume of a tumor. A further significant advantage is the ability to access difficult lesions not possible by use of simple needs as they would access only straight forward lesions. Thus, the needle assembly provides for effective therapy.

It should also be understood that a tumor environment is complex, and a single agent may only be effective for a portion of the tumor. The needle assembly 20 shown and described allows for multimodality treatment in that more than one agent may be used. Moreover, the needle assembly 20 may be used to delivery not only one or more chemical agents but also other therapeutic energy modalities including laser energy. The needle assembly 20 is particularly well-suited for targeting focal lesions in solid organs like the liver, kidney, lung, or other organs. Moreover, the needle assembly 20 offers flexibility in targeting tissue as well as selecting one or more appropriate treatment modalities.

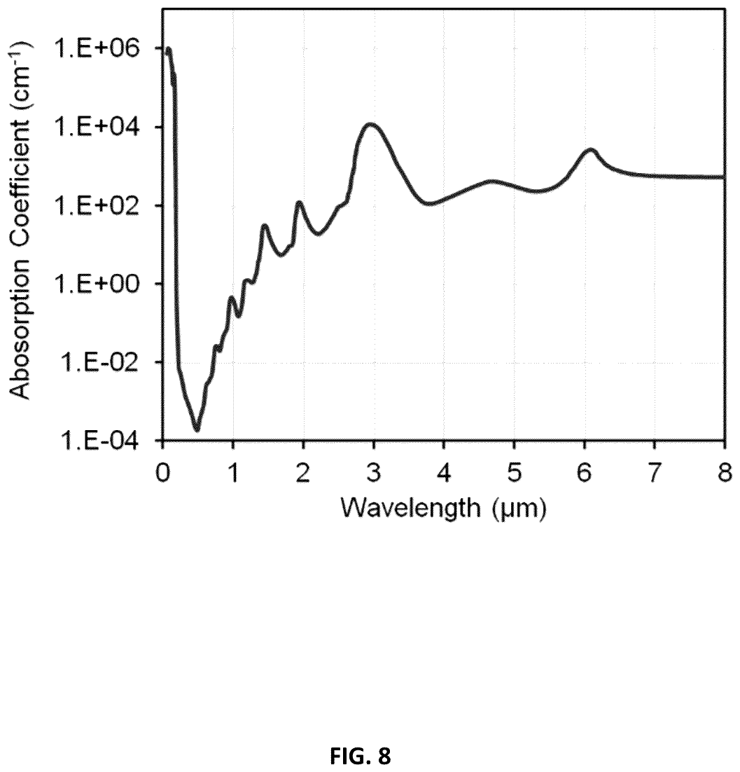

FIG. 7 illustrates one example of a delivery system 18. The delivery system 18 may include an enclosure 60 with one or more pump mechanisms disposed therein which may be used to delivery therapeutic agents from one or more fluid supplies 26 which may be fluidly connected to the needle assembly 20. Each of the pump mechanisms may be a programmable syringe-pump. The delivery system 18 may be configured to provide for injecting small amounts of drugs or other therapeutic agents such as a few microliters, or a few millimeters. Given the toxic nature of the drug or other therapeutic agent, the therapeutic agents are isolated, and a safe environment is provided which may be especially important where radiopharmaceuticals or live viral products are used. The delivery system 18 may also provide for injecting these amounts like a slow infusion so as not to oversaturate the tumor receptors. In some embodiments, tissue resistance may be tracked. In addition, as shown there may be multiple different amounts or types of therapeutic agents which may be delivered at the same time so that different parts of the tumor may be targeted.

Returning to FIG. 1, the control system 22 may be operatively connected to the delivery system 18 in order to control pump mechanism or other aspects of the delivery system 18. The control system 22 may provide for independently controlling each needle injection line as well as provide for storing, printing, displaying, communicating over a network, or otherwise conveying information regarding the use of the delivery system 18. The control system 22 may be operatively connected to a display 24 which is a touch screen display or other user interface to allow the user to control all of the injection parameters, review the parameters, or review other information such as displaying resistance to the injection, time into the injection, and time left for finishing the injection as well other available information regarding operation of the delivery system 18 or other connected aspects.

Returning to FIG. 7, the delivery system 18 may also provide for receiving energy from one or more other modalities such as laser energy. As shown in FIG. 7, coupling 14 may include a plurality of couplers which may be operatively connected to one or more lasers such as MIR lasers. One or more of the fluid supplies 26 may be replaced with fibers which may then extend through the needle assembly 20 to allow for delivery of both laser energy and chemical agents at the same time in a coordinated manner to provide multimodal therapy.

Although it to be understood that different types of surgical ablation therapies may be used including radiofrequency (RF), microwave (MW), irreversible electroporation (RE), and cryoablation, laser thermal ablation (LTA) therapy may be used. Some of the limitation associated with clinical use of LTA include the high cost and large footprint of the high-power surgical laser systems, the strict safety precautions needed when high-power lasers are used in an operating room, and the need for multiple treatments due to the small laser spot size that sometimes cannot remove all of the tumor in one treatment.

Among surgical lasers, ones operating in the ultraviolet (UV) (λ₀=0.19 to 0.40 μm), such as and near-IR (NIR) (λ₀=0.7 to 2.5 μm) wavebands are commonly utilized in surgical procedures. UV light has enough energy to break bonds in molecules that are building blocks of tissue resulting in lower risk in thermal damage to healthy tissue. UV light only penetrates hundreds of nanometers so are not suitable for tumor volume ablation therefore UV laser sources, such as bulky and power hungry excimer lasers are primarily used for ocular procedures such as LASIK. Furthermore, UV LTA often generates gaseous by-products which are undesirable for laparoscopic procedures, such as pulmonary vein isolation (PVI) ablation. Alternatively, NIR LTA surgeries rely on NIR light probing the strong absorption signatures of water molecules in the target tissue, resulting in heating of the tissue followed by ablative necrosis.

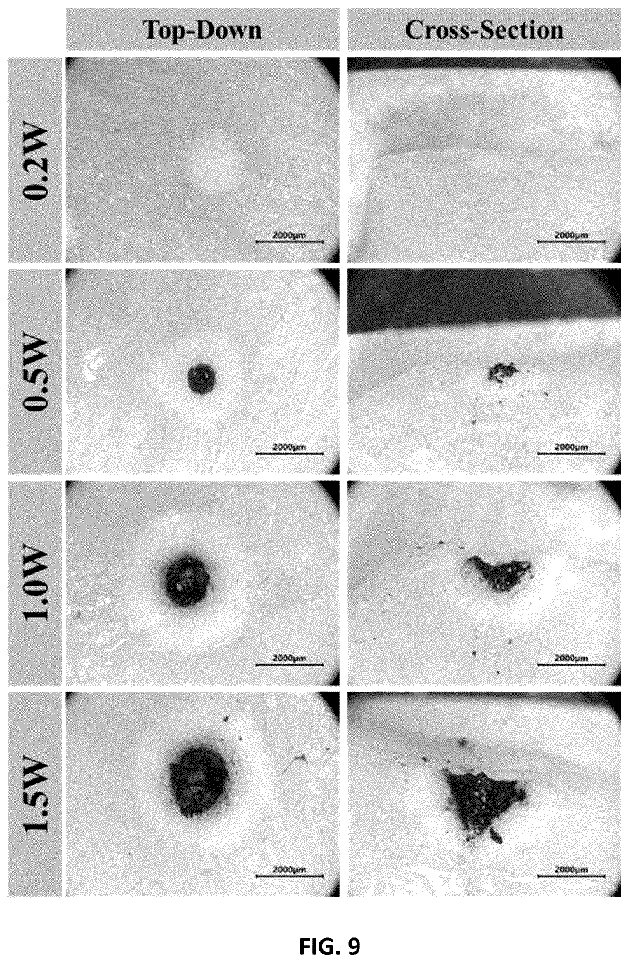

FIG. 8 shows the absorption coefficient of water as a function of wavelength obtained from D. J. Segelstein, [The complex refractive index of water] University of Missouri-Kansas City, (1981). Based on the water absorption coefficient in NIR wavelengths, NIR light can penetrate into tissue a few millimeters resulting in invasive thermal damage, where during NIR LTA damage of healthy tissue is unavoidable and can be problematic if performed near sensitive sites, such as nerves.

Mid-infrared (MIR) waveband (λ₀=3-8 μm) (A. D'Amico, C. D. Natale, F. L. Castro et al., “Volatile compounds detection by IR acousto-optic detectors.” 21-59) is an attractive spectral region for surgical ablation of soft tissues because building blocks of biological tissue, such as water, proteins, and lipids, exhibit molecular vibrational modes in the MIR wavelengths that result in strong absorption of laser light, supporting precise, minimally invasive procedures. For example, FIG. 8 confirms that water absorption is orders of magnitude higher in the MIR waveband (λ₀=3-8 μm) than the NIR band (λ₀=0.7 to 2.5 μm). This high absorption of MIR light results in hundreds of microns tissue penetration supporting precise excision with the possibility of selective tumor tissue ablation (E. Larson, M. Hines, M. Tanas et al., “Mid-infrared absorption by soft tissue sarcoma and cell ablation utilizing a mid-infrared interband cascade laser,” Journal of Biomedical Optics, 26(4), 043012 (2021)). However, mature MIR laser technologies for surgical applications are bulky light sources, such as the free electron lasers (Y. Xiao, M. Guo, K. Parker et al., “Wavelength-Dependent Collagen Fragmentation during Mid-IR Laser Ablation,” Biophysical Journal, 91(4), 1424-1432 (2006)), nonlinear crystals based optical parametric oscillators (OPOs), (V. S. Serebryakov, É. V. Boïko, A. G. Kalintsev et al., “Mid-IR laser for high-precision surgery,” Journal of Optical Technology, 82(12), 781-788 (2015); G. Edwards, M. S. Hutson, S. Hauger et al., [Comparison of OPO and Mark-III FEL for tissue ablation at 6.45 um] SPIE, PWL (2002)) and Strontium (Sr) vapor lasers, (M. Mackanos, B. Ivanov, A. Soldatov et al., [Ablation of soft tissue at 6.45 um using a strontium vapor laser] SPIE, PWB (2004)) among others. These MIR lasers are typically housed either in separate rooms (such as the basement of the hospital building where the operating room is located) or in large containments within the surgical room for safety reasons. The MIR laser radiation from the other rooms or containments is brought to the surgical bed using optical fibers that experience bending and transmission losses and as a result may not provide the freedom of motion that is necessary in surgical procedures. The bulky laser systems also occupy a large footprint within the surgical room, which is not ideal in already crowded operating rooms (ORs). Due to these challenges, MIR LTA is still an exploratory surgical technology.

Recent advances in quantum engineered materials have made quantum cascade lasers (QCLs) (J. Faist, F. Capasso, D. L. Sivco et al., “Quantum cascade laser,” Science, 264(5158), 553-556 (1994)) that are MIR emitters commercially available. QCLs have miniature footprint (˜10 μm by ˜100 μm by ˜3 mm), operate at room temperature and are power efficient making them promising for precise surgical ablation procedures, such as PVI. However surgical ablation using QCLs has not been broadly researched (P. I. Abramov, E. V. Kuznetsov, L. A. Skvortsov et al., “Quantum-Cascade Lasers in Medicine and Biology (Review),” Journal of Applied Spectroscopy, 86(1), 1-26 (2019)). Little to no work has been done to demonstrate their safety and efficacy for photothermal therapies, such as ablation. In 2012, Huang and Kang (Y. Huang, and J. U. Kang, “Corneal tissue ablation using 6.1 μm quantum cascade laser.” 8209, 167-172) performed a bovine corneal ablation test using a pulsed QCL (λ₀=6.1 μm). Their preliminary results demonstrated successful ablation even when limited by the minimum pulse width (5 ms) and laser power (Pₘₐₓ=792 mW). In 2014, Hashimura et. al. (K. Hashimura, K. Ishii, N. Akikusa et al., “Coagulation and ablation of biological soft tissue by quantum cascade laser with peak wavelength of 5.7 μm,” Journal of Innovative Optical Health Sciences, 07(03), 1450029 (2014)) evaluated a high-power QCL (λ₀=5.7 μm) for use as a laser scalpel. Their prototype QCL had a variable pulse width (20-500 ns) and repetition rate (1-1000 kHz), and an average pulse energy of either 0.495 or 0.990 μJ/pulse. They compared its performance to that of a conventional carbon dioxide (CO₂) laser, and found the two to have similar coagulation, carbonization, and ablation effects. Prior work (E. Larson, M. Hines, M. Tanas et al., “Mid-infrared absorption by soft tissue sarcoma and cell ablation utilizing a mid-infrared interband cascade laser,” Journal of Biomedical Optics, 26(4), 043012 (2021)) used a λ₀=3.3 μm interband cascade laser (ICL), where the dominant chromophore is water, demonstrating that λ₀=3.3 μm wavelength is capable of ablating pleomorphic sarcoma tumor cells (C1619) at extremely low intensity values (93.75 mW/cm²) confirming that probing the water absorption in MIR can successfully enable cancer cell death even at low power doses.

Ablation of chicken breast tissue is demonstrated using a higher power commercially available λ₀=4.65 μm QCL (QF4650HHLH, Thorlabs, NJ, USA) with a maximum power output (Pₘₐₓ) of 1.5 W. The high power QCL comes with a high heat load (HHL) package that requires the use of a liquid-cooled mount (LCM100, Thorlabs, NJ, USA) that attaches to a tabletop liquid chiller (LK220, Thorlabs, NJ, USA). Experimental ablation of chicken breast tissue demonstrates the efficacy of MIR ablation by quantizing the energy dosage necessary to generate specific penetration depth and spot size during surgical procedures. The experimental relationships are further validated by finite element method (FEM) modeling via COMSOL Multiphysics (C. M. S. S.-www.comsol.com).

Both experimental ablation and FEM modeling were performed. The simulations were performed to validate the results observed from experimental ablation. The experimental ablation studies were performed using retail chicken breast. Further details on these procedures and data analysis can be found below.

In each study, laser parameters were varied to characterize their effect on the coagulated diameter and depth. In Study T, the laser power and exposure time were varied, and all ablations were performed at continuous wave (CW). In Study F, the laser power and quasi-continuous wave (QCW) pulse frequency were varied, and all ablations were performed for 30 seconds at 50% duty cycle. Lastly, in Study D the laser power and pulse duty cycle were varied, and all ablations were performed for 30 seconds at 20-Hz (pulses per second).

For all studies, chicken breast tissue was acquired from a local retailer. It was thawed using a conventional microwave oven and manually diced into appropriately sized pieces with a kitchen knife. Their surfaces were gently pressed level with the side of the kitchen knife, then ablated for one power (0.2, 0.5, 1.0, or 1.5 W, CW) and time setting (Table 1) in three different spots (n=3). The sample was then immediately imaged top-down with the same microscope and software. Then, an X-acto knife was used to carefully cut through the ablated spot without destroying it. Each spot was then imaged cross-sectionally with the same microscope and software.

TABLE 1
Chicken Ablation Studies - Parameter Summary Table. (A)
Study T parameters. These times are chosen so, because
as power increases the required time to achieve ablation
decreases. (B) Study F parameters. (C) Study D parameters.

| | Tissue Sample ID | Power (W) | Exposure Time (s) |
|---|---|---|---|
| Study T | T1 | 0.2 | 30, 60, 90, 120 |
| | T2 | 0.5 | 15, 30, 45, 60 |
| | T3 | 1.0 | 10, 20, 30, 40 |
| | T4 | 1.5 | 5, 10, 15, 30 |
| | Tissue Sample ID | Power (W) | Pulse Frequency (Hz) |
| Study F | F1 | 0.2 | 1, 10, 100, 1000 |
| | F2 | 0.5 | 1, 10, 100, 1000 |
| | F3 | 1.0 | 1, 10, 100, 1000 |
| | F4 | 1.5 | 1, 10, 100, 1000 |
| | Tissue Sample ID | Power (W) | Pulse Duty Cycle (%) |
| Study D | D1 | 0.2 | 15, 30, 50, 75 |
| | D2 | 0.5 | 15, 30, 50, 75 |
| | D3 | 1.0 | 15, 30, 50, 75 |
| | D4 | 1.5 | 15, 30, 50, 75 |

In the MIR LTA testing setup was configured for testing. In all ablation experiments, a 4.65-μm HHLH QCL (QF4650HHLH, Thorlabs, NJ, USA) was attached to a liquid-cooled mount (LCM100, Thorlabs, NJ, USA). The laser pins were connected to the mount which was connected to a driver/controller (ITC4005QCL, Thorlabs, NJ, USA) via HHLH connector cables (CAB4007A, Thorlabs, NJ, USA). The laser mount was connected via polyurethane hosing (HPU6, Thorlabs, NJ, USA) and fittings (QVF6 & QVM6, Thorlabs, NJ, USA) to a water cooled 200 W capacity chiller (LK220 Thorlabs, NJ, USA), which cooled the refrigerant (CDTX, Thorlabs, NJ, USA) and pumped it through the hosing and mount. An electronically controlled shutter was held directly below the lens aperture and operated via shutter controller (SC10 Thorlabs, NJ, USA) for precise exposure timing. In all ablation trials, before performing chicken ablation, the drive current required to achieve the desired output power was found by measuring the output power with a photodetector (PM16-401, Thorlabs, NJ, USA).

After ablation, the tissue showed three different morphological changes. In increasing severity of ablation, the region would appear (1) white and coagulated resembling cooked chicken, (2) dark-brown and charred, (3) completely ablated and devoid of the tissue. The surface diameter and penetration depth of the coagulation and charring were used as metrics to characterize the extent of ablation. Ablated regions were imaged using an optical microscope (AmScope, 2054729). Images were acquired with ToupView imaging software (v. x64, 4.11.19728.20211022). Top-down images were taken first to measure the surface diameter, and cross-sectional images were taken after to measure the coagulated and charred depth. Samples were prepared for cross-sectional imaging by slicing through the ablated spot using an Exacto™ razor.

To complement the comparison of coagulation between experimental and modeled results, thermographic videos were also recorded to compare the maximum temperatures. The FLIR ONE Edge pro (FLIR Teledyne) was selected for its temperature range (−20° C.-120° C. and 0° C.-400° C.), mobility, device compatibility, and affordable cost compared to expensive, higher resolution cameras. In these recordings, the camera was held 5 to 10-cm from the ablated spot on the tissue surface. This was the closest possible distance without interrupting the path of the laser beam. The maximum temperature was annotated during the recordings, which allowed for later analysis and comparison.

The photothermal ablation of chicken breast was simulated using COMSOL to validate the results from experimental tissue sample ablation. This model accounts for MIR (λ₀=4.65 μm) ray propagation and absorption within the tissue, and its resultant heat generation, heat transfer, and thermal damage.

The simulated laser had a vacuum wavelength of λ₀=4.65-μm, an incident spot diameter of 2-mm, a Gaussian intensity profile and total power of 0.2-W. Optical and thermal properties were required to accurately model the tissue ablation. However, these are dependent on temperature and water-content, making tissue ablation a dynamic process. At high laser powers, the water in the tissue would boil and the tissue itself would burn. The dynamics of these phenomena are especially difficult to accurately model, and for that reason only the 0.2-W ablation was modeled.

The various material properties of chicken breast are dependent on water content (R. Y. Murphy, and B. P. Marks, “Apparent thermal conductivity, water content, density, and porosity of thermally-processed ground chicken patties”, Journal of Food Process Engineering, 22(2), 129-140 (1999)) and temperature. The densities, heat capacities, and thermal conductivities of liquid water and steam were taken from COMSOL's built-in material properties for each. The refractive index of the tissue is approximated by that of water (R. Y. Murphy, and B. P. Marks, “Apparent thermal conductivity, water content, density, and porosity of thermally-processed ground chicken patties,” Journal of Food Process Engineering, 22 (2), 129-140 (1999); G. M. Hale, and M. R. Querry, “Optical Constants of Water in the 200-nm to 200-μm Wavelength Region,” Applied Optics, 12(3), 555-563 (1973)). The extinction coefficient of the tissue is expressed as that of water multiplied by the water content fraction while the temperature-dependent density, heat capacity, and thermal conductivity of tissue are expressed as linear sums of the thermal properties of dry tissue, water, and steam, weighted by the temperature-dependent water content. The bioheat parameters blood perfusion and metabolic heat source were chosen to be zero, because there is none present during the in vitro ablation experiments. Various values for the Arrhenius thermal damage model parameters, activation energy (Eₐ=455 KJ/mol) and frequency constant (A=7.66E66 s⁻¹), were found in literature.

The coagulated diameter and depth were determined using the damaged fraction parameter (θ), which varied from zero (undamaged) to one (completely coagulated). The damaged fraction was calculated from the Arrhenius thermal damage model. Cut lines were placed across the surface of the tissue, and from the surface down into the tissue to evaluate the damaged fraction diameter and depth profiles. Coagulated diameter was calculated similarly to full-width half-max (FWHM), from where θ crossed 0.5 on one side to the other. Coagulated depth was calculated as the distance from the surface to the point where θ again crossed 0.5.

Following, the results of the experimental ablation and FEM simulation can be found. Microscopic images show how the ablated spots appeared to the naked eye. From such images the diameters and depths of the ablated regions were measured, and these results are summarized below. FEM simulation shows the evolution of the temperature profile during the ablation and shows final temperature and coagulation profiles which are comparable to the equivalent experimental result

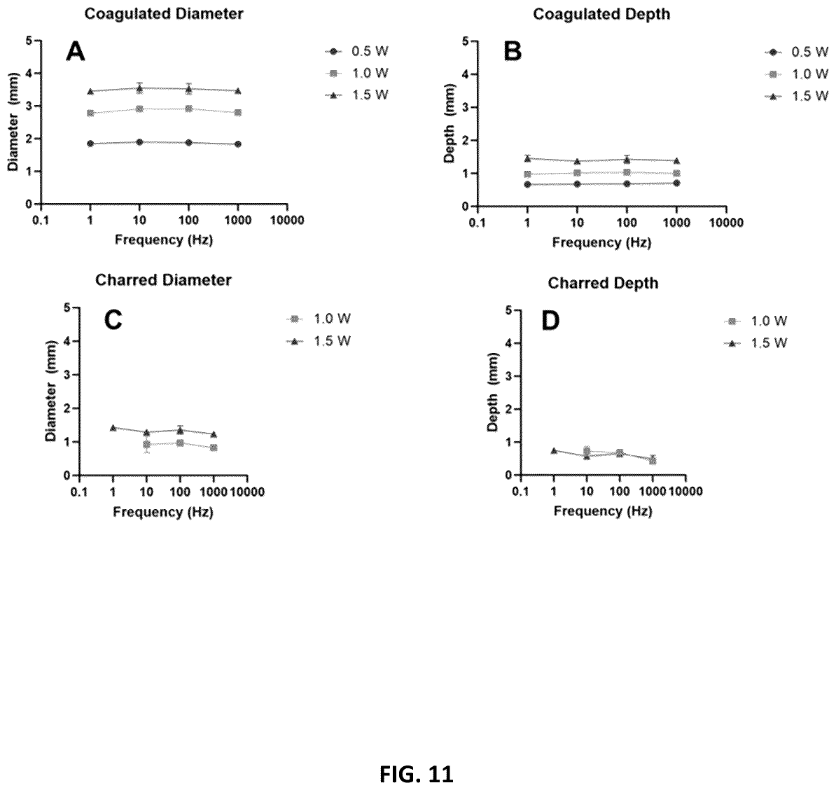

FIG. 10 shows the growth of the coagulated and charred diameter and depth over time for each of the four tested CW laser power settings (0.2, 0.5, 1.0, 1.5 W) in Study T (n=3). The results from semi-log regression of these data are listed in Table 2. In all subfigures, the coagulated and charred diameter and depth generally increase with power. However, the coagulated and charred depths (B and D) seem to saturate when ablated at 1.0-1.5 W. Similarly, while the regression slopes in A generally increase with power, they also seem to saturate at 1.0 W in B and D. The high R² values in the coagulation subfigures (A and B) generally indicate a high goodness-of-fit. In subfigures C and D, there is no 0.2 W data, because 120-sec of exposure was insufficient to cause charring at this relatively low power. They also show that charring can be observed at 0.5, 1.0, and 1.5 W at a dose of 7.5, 5.0, 1.5 J, respectively. For these charring data, the regression slope generally increases with power, and the R² value indicates high goodness-of-fit. The exception is at 0.5 W, which is caused by the high data variance and the method by which R² is calculated.

TABLE 2
Study T - Parameters of semi-log regression fits.

| Study F | | | | | | | | |
|---|---|---|---|---|---|---|---|---|
| Semilog | Coagulated Diameter | | | | Coagulated Depth | | | |
| Regression | 0.2 W | 0.5 W | 1.0 W | 1.5 W | 0.2 W | 0.5 W | 1.0 W | 1.5 W |
| Slope | — | −0.007 | 0.004 | 0.002 | — | 0.013 | 0.009 | −0.013 |
| Y-intercept | — | 1.877 | 2.849 | 3.498 | — | 0.664 | 0.997 | 1.433 |
| P | — | 0.671 | 0.833 | 0.934 | — | 0.100 | 0.531 | 0.534 |
| Study F | | | | | | | | |
| Semilog | Charred Diameter | | | | Charred Depth | | | |
| Regression | 0.2 W | 0.5 W | 1.0 W | 1.5 W | 0.2 W | 0.5 W | 1.0 W | 1.5 W |
| Slope | — | — | −0.025 | −0.053 | — | — | −0.124 | −0.068 |
| Y-intercept | — | — | 0.972 | 1.410 | — | — | 0.882 | 0.721 |
| P | — | — | 0.795 | 0.030 | — | — | 0.118 | 0.012 |

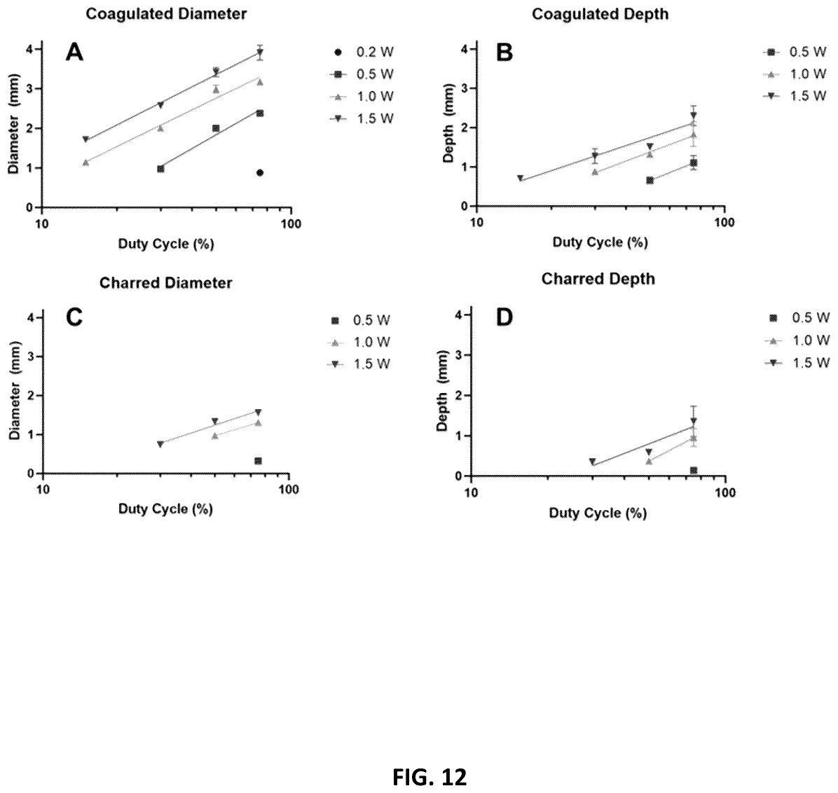

FIG. 11 shows the final coagulated diameters and depths of tissue ablated in Study F. The best-fit parameters of semi-log regression are shown in Table 3. Which also shows the P values from testing that the best-fit slope is statistically different from zero. Values closer to zero indicate more significant difference from zero. All ablations were performed for 30-sec at 50% duty cycle. No 0.2 W data is shown in any subfigures because no visible coagulation was observed after 30 seconds at 0.2 W for any QCW frequency. The coagulated diameter and depth increase with the laser output power. At this duty cycle (50%) and exposure time (30-sec), both 1.0 W and 1.5 W ablations caused charring, but 0.5 W did not. The charred diameter is also larger for 1.5 W than 1.0 W, but there is no significant difference in charred depth between the two powers, which agrees with FIG. 10, panel D. Generally, there is no significant trend in coagulated or charred diameter or depth with QCW pulse frequency because the regression slopes were found to be not significantly different from zero. The exceptions to this are the 1.5 W charred diameter and depth.

TABLE 3
Study F - Parameters of Semi-Log Regression. The slope and y-intercept
are best-fit parameters from semi-log regression, and the P value
tests the null hypothesis that the overall slope is zero.

| | Coagulated Diameter | | | | Coagulated Depth | | | |
|---|---|---|---|---|---|---|---|---|
| Study T | 0.2 W | 0.5 W | 1.0 W | 1.5 W | 0.2 W | 0.5 W | 1.0 W | 1.5 W |
| Slope | 0.7720 | 1.347 | 1.418 | 1.653 | 0.3529 | 0.8804 | 1.242 | 1.174 |
| Y-intercept | 0.6904 | 1.084 | 1.734 | 1.865 | −0.0219 | 0.0402 | 0.2814 | 0.4006 |
| R² | 0.9473 | 0.9455 | 0.9736 | 0.9521 | 0.9553 | 0.8097 | 0.8539 | 0.8572 |
| | Charred Diameter | | | | Charred Depth | | | |
| Study T | 0.2 W | 0.5 W | 1.0 W | 1.5 W | 0.2 W | 0.5 W | 1.0 W | 1.5 W |
| Slope | — | 0.6948 | 0.6008 | 0.9396 | — | 0.2725 | 0.8021 | 0.7505 |
| Y-intercept | — | −0.1563 | 0.6371 | 0.6284 | — | −0.0157 | −0.0998 | 0.1030 |
| R² | — | 0.4149 | 0.9228 | 0.9557 | — | 0.3558 | 0.7330 | 0.7747 |

FIG. 11 shows the final coagulated diameters and depths of volumes ablated in Study D. All ablations were performed at 20-Hz for 30-sec. Data for 0.2 W is absent from most subfigures because this power setting was insufficient to generate measurable coagulation or charring. The coagulated and charred diameter and depth all increase with laser power and duty cycle. Table 4 shows the parameters of semi-log regression applied to the data in FIG. 11. The slopes are similar within each metric, except for the charred depth. The y-intercepts consistently increase within each metric as power increases, with the exception of charred diameter. In all metrics, the r-squared value shows a strong correlation between duty cycle and said metric.

TABLE 4
Study D - Parameters of semi-log regression fits.

| Study D | | | | | | | | |
|---|---|---|---|---|---|---|---|---|
| Semilog | Coagulated Diameter | | | | Coagulated Depth | | | |
| Regression | 0.2 W | 0.5 W | 1.0 W | 1.5 W | 0.2 W | 0.5 W | 1.0 W | 1.5 W |
| Slope | — | 3.593 | 3.068 | 3.191 | — | 2.537 | 2.375 | 2.116 |
| Y-intercept | — | −4.268 | −2.451 | −2.065 | — | −3.657 | −2.656 | −1.847 |
| R² | — | 0.9615 | 0.9683 | 0.9843 | — | 0.7910 | 0.8549 | 0.8832 |
| Study D | | | | | | | | |
| Semilog | Charred Diameter | | | | Charred Depth | | | |
| Regression | 0.2 W | 0.5 W | 1.0 W | 1.5 W | 0.2 W | 0.5 W | 1.0 W | 1.5 W |
| Slope | — | — | 1.909 | 2.085 | — | — | 3.298 | 2.451 |
| Y-intercept | — | — | −2.269 | −2.296 | — | — | −5.23 | −3.365 |
| R² | — | — | 0.9808 | 0.9515 | — | — | 0.8317 | 0.7374 |

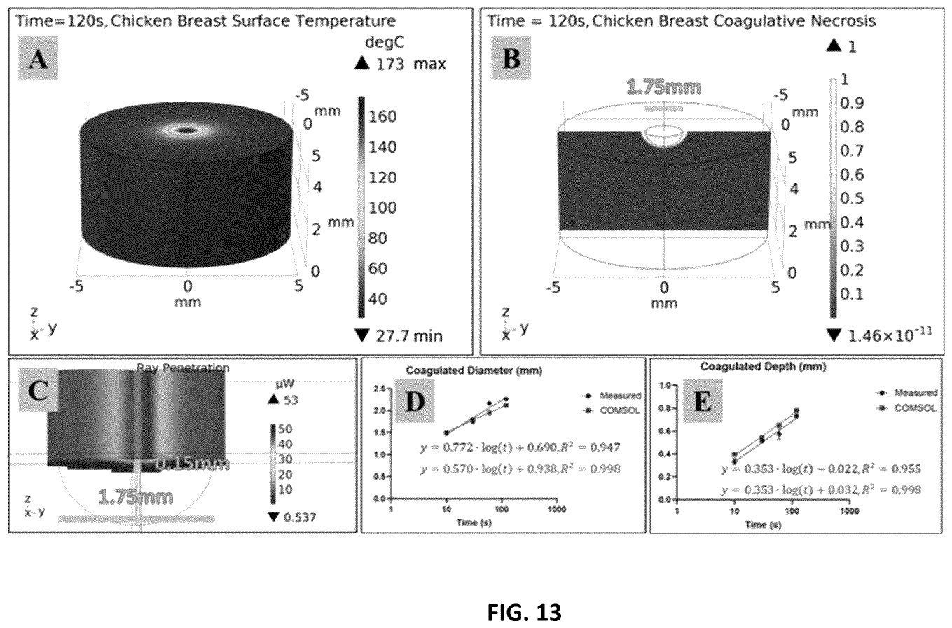

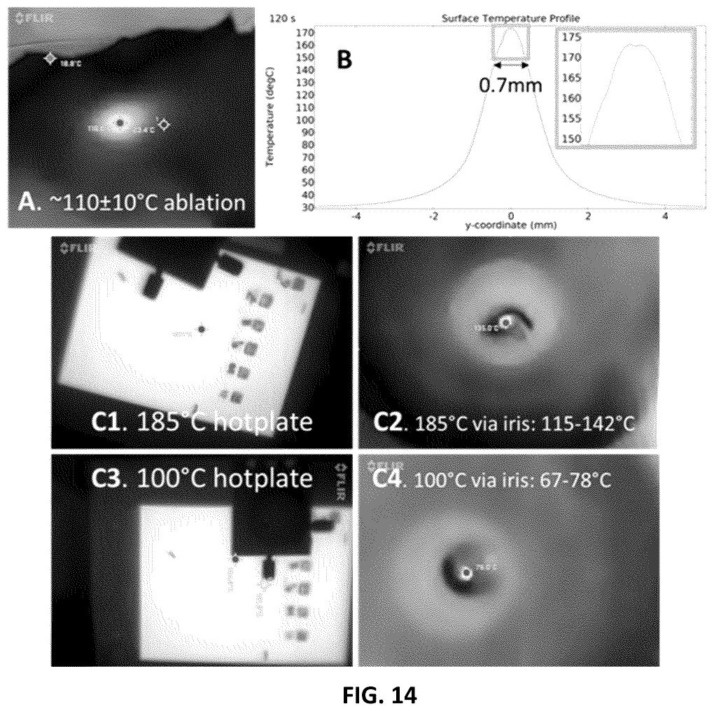

FIG. 12 shows the results of numerically modeling chicken breast ablation with a 2-mm, CW 0.2-W Gaussian laser beam (λ₀=4.65 μm) for 120 seconds. Subfigure A shows the surface temperature of the chicken after 120-sec. The chicken started at 20° C. and reached a minimum and maximum temperature of 27.7° C. and 174° C., respectively. B shows a profile of the coagulative necrosis in the y-z plane. The white hemi-circle at the top represents the coagulated region and the red area is uncoagulated. C shows the trajectories of the laser's rays and their penetration into the surface of the chicken tissue. The maximum and minimum ray powers are 53 and 0.53-μW, respectively. Therefore, the maximum penetration depth is 0.15-mm, because by that depth the ray power has been >99% absorbed. D shows how the depth of the coagulative necrosis increases over time and compares the experimentally measured and numerically simulated values. Comparing the semi-log regression between the measured and COMSOL coagulated diameter data, the slopes are not significantly different (P=0.0797) and neither are the y-intercepts (P=0.1439). E shows how the coagulated diameter grows over time, and similarly compares measured and simulated values. Comparing the semi-log regression between the measured and COMSOL coagulated depth data, the slopes are not significantly different (P=0.9997). The y-intercepts are significantly different (P=0.0079), but they are both very close to 0-mm.

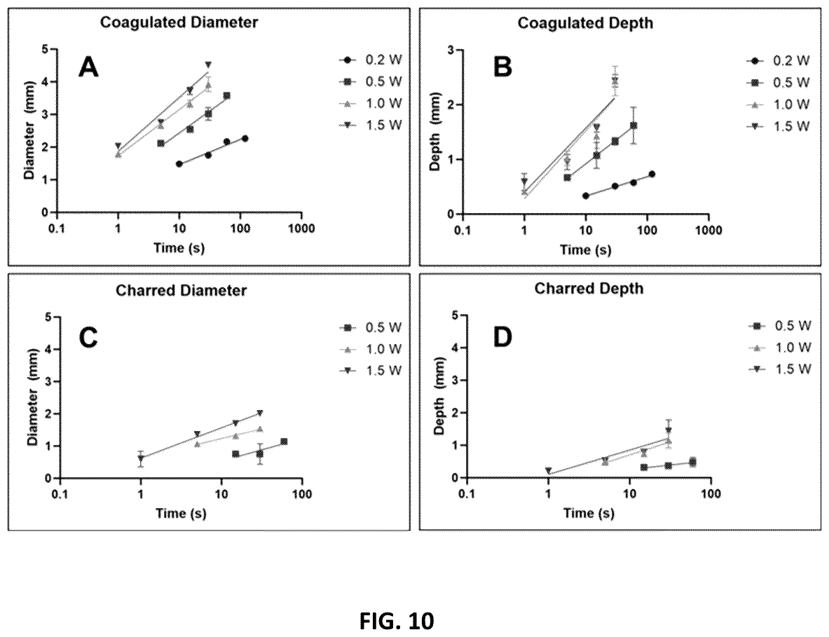

During the duration of the laser exposure, the surface of the chicken can be seen progressing from a healthy pink and orange color to coagulated white and charred dark brown (FIG. 9). These observations serve as a visible indication of the thermal damage process underway. The whitening of tissue indicates the denaturation of hemoglobin and myoglobin which is known to occur at temperatures between 55-80° C. This protein denaturation has also been studied and characterized using Arrhenius kinetics, from which values of activation energy were found and used in the numerical modeling. At temperatures above 100° C., water evaporates from the tissue causing bubbles of steam to form and expand near the surface. At around 400° C., the chicken starts to burn and char. At this point, the water which composed much of the volume has entirely evaporated and the remaining protein content and other constituents have suffered severe damage turning dark brown. At this point a cavity can be seen at the ablated point, where all the water has evaporated, and the charred protein coats the tissue-air boundary. This process is more rapid for higher power settings, as more energy is absorbed by the water causing it and the tissue to heat up faster. Eventually, the maximum temperature saturates as the diffusive thermal energy flux balances the laser heat source. At higher power settings, more energy is absorbed, the maximum temperature is higher, and the extent of ablation is greater.

In these ablation experiments, the optical properties of the tissue determine how the light penetrates or is reflected and is absorbed or scattered. The mechanical and thermal properties determine how thermal energy flows through the tissue. At different temperature and energy thresholds, coagulation, evaporation, charring, and other such changes occur affecting these properties. Also, all these properties depend strongly on the water content of the tissue which is also dependent on its temperature. Based on numerical and experimental observations, the maximum temperature and experimental metrics of ablation obey an asymptotic relationship with time. Multiple different forms of regression were compared for these measurements, and semi log regression (Eq. 1) was found to fit with the best Pearson R² value.

$$y = m log ( x ) + b \qquad ( 1 )$$

Where y is the measured ablation metric, m is the slope, x is the independent variable (Study T: time; Study F: QCW frequency; Study D: QCW duty cycle), and b is the y-intercept. Though this is an over-simplification of the dynamic physical processes occurring during ablation, it was found sufficient for comparing these results.
a) In the results of Study T (FIG. 10, Table 2), these positive trends between laser power and exposure time can be seen. Data with high variance are best explained by variations in local tissue composition and the methods employed for measuring ablated depth. This involved cutting through the ablated spot with an Exacto™ razor blade to image the cross-section, which could cause minor deformations in the tissue. Comparing the regression fits in FIG. 10 and Table 2, the slopes indicate that higher power causes a faster ablation rate. The y-intercepts also show a positive relationship with laser power. This trend is a result of the lower duration threshold required to cause measurable ablation for higher powers. Ideally, all y-intercepts should be zero, because no coagulation should occur after zero seconds. All R² values show strong correlation between ablation time and diameter or depth, except for the 0.5 W cases of charred diameter and depth. These exceptions are likely due to the weak slope and high variance. Similarly, the high-power settings often have the strongest R² values because they have stronger slopes and lower variances. Looking at FIG. 10, panels B, D, the depths don't increase much from 1.0 W to 1.5 W and the slopes are not significantly different (p=0.9827). This suggests that there is a laser power saturation point where no further increase in power yields an increase in the extent of ablation. This may be due to the tissue's reduced ability to absorb light once it's charred. The onset of charring in C and D varies with power, which implies that dose, calculated as total power multiplied by time, is not a reliable metric for predicting onset of charring. This may be due to the dynamic nature of the ablation process, which is discussed above. Consider that the diameter of the incident laser beam is 2-mm and the absorption coefficient for water at λ₀=4.65 μm is α=410.77 cm⁻¹. According to the Beer-Lambert Law, and assuming tissue is 75% water, 99% of the beam's energy should be absorbed and generate heat within the first 150-μm of the surface (Eq. 2).

$$I ( z ) = {I}_{0} {e}^{- α z} ∴ z = \frac{ln ( 0.01 )}{- 0.75 \cdot 410.77} ≅ 1 49.5 μm \qquad ( 2 )$$

Where I(z) is the beam intensity as a function of tissue depth, and I₀ is the initial beam intensity. The ablation which occurs within the surrounding few millimeters of the absorbing volume is due to this generated thermal energy diffusing away into the surrounding tissue and damaging it. In the case of the 0.2 W trials, no charring was observed for any exposure duration, the coagulated diameter grew from 1.28-mm to the approximately the size of the incident beam (2.03-mm), and the depth grew from 0.26-mm to 0.90-mm over 120-sec. This coagulation depth is much more than the penetration depth of the laser, which is likely due to the diffusion of thermal energy away from the absorbing volume of tissue. Accepting that the CW laser power threshold for charring is low for this beam size (<0.5 W), it is possible to cause spatially precise coagulative necrosis in chicken breast while avoiding charring.

In the results of Study F (FIG. 11), there exists a clear positive relationship between power and the ablation metrics, just as in Study T. Higher power laser settings cause a greater extent of ablation. In these trials, where duty cycle was kept at 50% and all ablations were performed for 30-sec, there is no significant relationship between QCW pulse frequency and any ablation metric, as shown by the P values in Table 3. For any given power setting and duty cycle, the pulse-averaged power (power multiplied by fraction of time on) is independent of pulse frequency. In these trials, no visible coagulation occurred at 0.2 W for any frequency setting, because the 30-sec duration and duty cycle were not sufficient to cause significant heating. Similarly, no 0.5 W data is present in C or D because the duration and duty cycle were insufficient to cause charring. Comparing the charred depths at 1.0 and 1.5 W, no significant difference can be seen between their slopes (p=0.9991). This agrees with the equivalent data in Study T, further suggesting a power saturation level. Although the power settings 1.0 and 1.5 W are high enough to cause charring for this laser beam size, it is possible to achieve spatially precise ablation with 0.5 W and avoid charring. Moreover, the extent of both coagulation and charring are highly precise relative to the laser beam diameter and penetration depth.

In the results of Study D (FIG. 12, Table 4), all ablation trials were performed for 30-sec at a pulse frequency of 20-Hz using the same gaussian laser beam with a 2-mm diameter and 24.3 μm penetrations depth in water. This pulse frequency was chosen to allow for sufficient thermal relaxation according to the thermal relaxation constant (Tr) which can be calculated according to Eq 3 below.

$${τ}_{r} = \frac{{δ}^{2}}{4 α} = \frac{1}{4 {αμ}_{a}^{2}} ∴ {τ}_{r} ( 4.65 μm ) = \frac{1}{4 \cdot 1.42 \times {10}^{- 3} \cdot {( 0.75 \cdot 410.77 )}^{2}} ≅ 1.8 \; 55 ms \qquad ( 3 )$$

Where δ (cm) is the skin depth which is the inverse of the absorption coefficient ula (cm⁻¹), and α is the thermal diffusivity (cm²/s). Similarly to the results from Study T, these results were best fit by semi-log regression. In each subfigure, all the slopes are very similar to each other which shows that the dependence of ablation rate on duty-cycle is independent of laser power. In contrast, the y-intercepts have a significant positive correlation with power. This is again because higher power lowers the duration threshold for coagulation and generally increases the extent of ablation. There is only one data point for 0.2 W in A and for 0.5 W in C and D, because only at 75% duty cycle was there sufficient heating to cause coagulation or charring, respectively.

The coagulated diameter and depth calculated from the COMSOL model are in excellent agreement with those from experimental ablation. The tissue's properties are defined as weighted sums dependent on temperature and water content which estimates the dynamic optical and thermal phenomena during the ablation process. These property values are taken from literature.

The coagulated depths from numerical modeling slightly overestimate the experimental coagulated depths. These differences are small enough that they may be explained by low sample number (n=3). In contrast, the modeled coagulated diameters qualitatively appear to slightly underestimate the experimental measurements. Though in this case the differences in the parameters of best-fit regression are not statistically significant, as mentioned above.

Maximum temperatures in the model do not agree with those reported by the thermography camera (FIG. 8, panels A-B). However, the camera used is limited in the size of the spot it can resolve and how accurately it reports the spot's temperature when it is much higher than the surrounding area. To account for this limitation of this camera, a conversion factor was calculated to correct for the temperature discrepancy. The camera was used to measure the temperature of a hotplate from two perspectives: (1) through the aperture of an adjustable optical iris, and (2) directly viewing the hotplate without the iris. When viewing the hotplate through the aperture, the iris was closed to its minimum diameter (0.7-mm) which was approximately the size of the hotspot as reported by COMSOL. Additionally, the camera was held away from the iris aperture by 5-cm which was the same distance the camera was held from the sample during ablation. By comparing the camera's maximum detected temperature in these two perspectives, a conversion ratio was calculated.

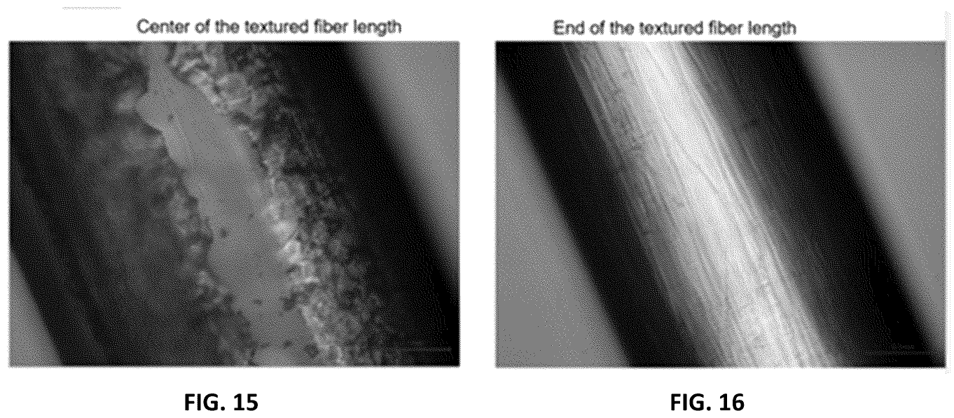

FIG. 14 shows (A) the modeled temperature profile across the surface and through the center of the ablated spot after 120-sec. This shows that the average center temperature within a 0.7-mm diameter is >165° C. In B, thermographic images of the tissue surface show the temperature after 120-sec of ablation. In these images the camera measures a maximum temperature of 110±10° C. which disagrees with the model's maximum temperature of 173° C. In C, multiple camera images show the temperature of the hotplate, set to 100° C. and 185° C., from both perspectives with and without the iris. These measurements show that the temperature through the iris is inaccurate by a factor of 0.7±0.08 compared to the true hotplate temperature. This factor accounts for the maximum temperature discrepancy between the thermographic camera and model's results, overcomes the limitations of the camera, and further validates the COMSOL model by proving its maximum temperatures are accurate to experimentally measured values.

As shown above 4.65-μm QCL may be used photothermal ablation. Using benchtop experimentation and COMSOL FEM validation, the efficacy and spatial precision of this ablation technology is validated by quantifying the width and depth of the ablated volume across various laser powers, exposure times, and QCW frequencies and duty cycles. These results show the viability of MIR QCLs for precision photothermal surgical ablation.

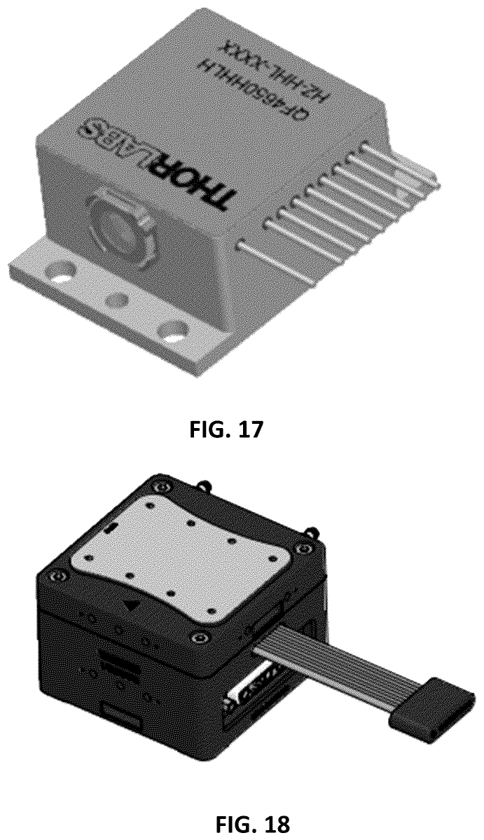

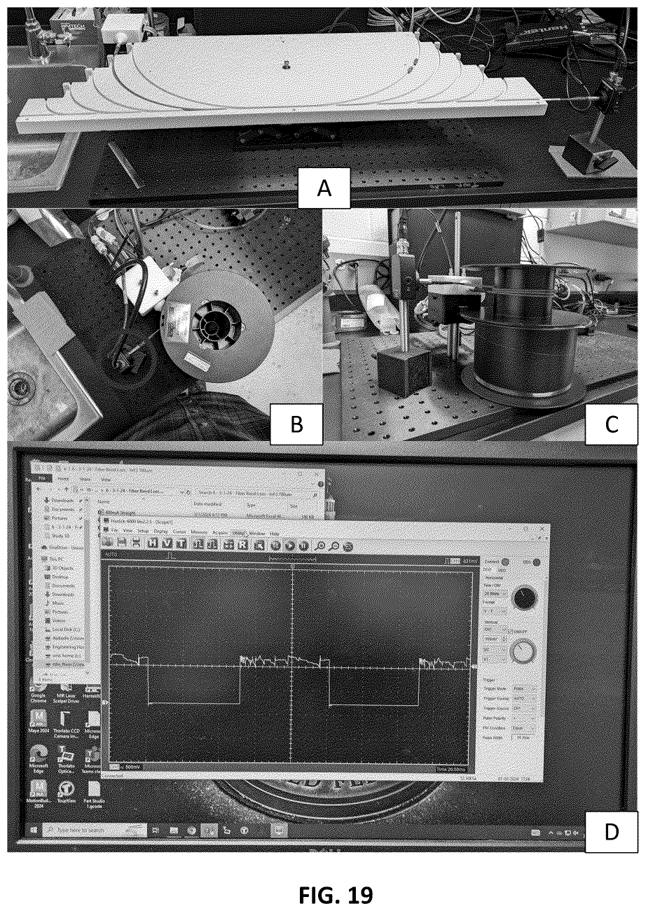

Returning to FIG. 1 the fibers used may be MIR transparent fibers 16. The MIR transparent fibers may be fluoride fibers which are fibers based on fluorides such as indium fluoride (InF3) or zirconium fluoride (ZrF₄). The MIR transparent fibers may be textured for diffused light output. FIG. 15 and FIG. 16 illustrate one example of textured MIR transparent fibers.

The MIR transparent fibers may be textured in any number of ways. One way of texturing the MIR transparent fibers is through etching such as a piranha etch, or a dilute piranha etch where a chemical mixture is used to etch the surface. In an acidic piranha etch, sulfuric acid (H₂SO₄) and hydrogen peroxide (H₂O₂) may be used. In a basic piranha etch, ammonium hydroxide (NH₄OH) may be used instead of sulfuric acid and this may be mixed with hydrogen peroxide and water. It is to be further understood that any effective concentrations of the components may be used. Of course, any number of other types of etching with any number of other chemicals may be used. For example other acids such as, but not limited to, hydrochloric acid (HCl), nitric acid (HNO₃), may be used. Alternatively, fluoride fiber texturing may be performed using polishing paper, since fluorides are soft materials and may be textured via polishing paper such as polishing paper with 9, 15, and 30 micron grit. For example, a 15-micron grit polishing paper may be used to polish a 200 μm core InF3 fiber. The texturing of fluoride tip fibers assists in providing diffused light output.

Returning to FIG. 1, coupling 14 is shown. The laser-fiber coupling is preferably performed in a manner which results in reduced loss and also allows the fibers to be bendable. Mid-IR laser coupling optics may use a combination of mid-IR transparent lenses. Preferably the coupler reduces losses.

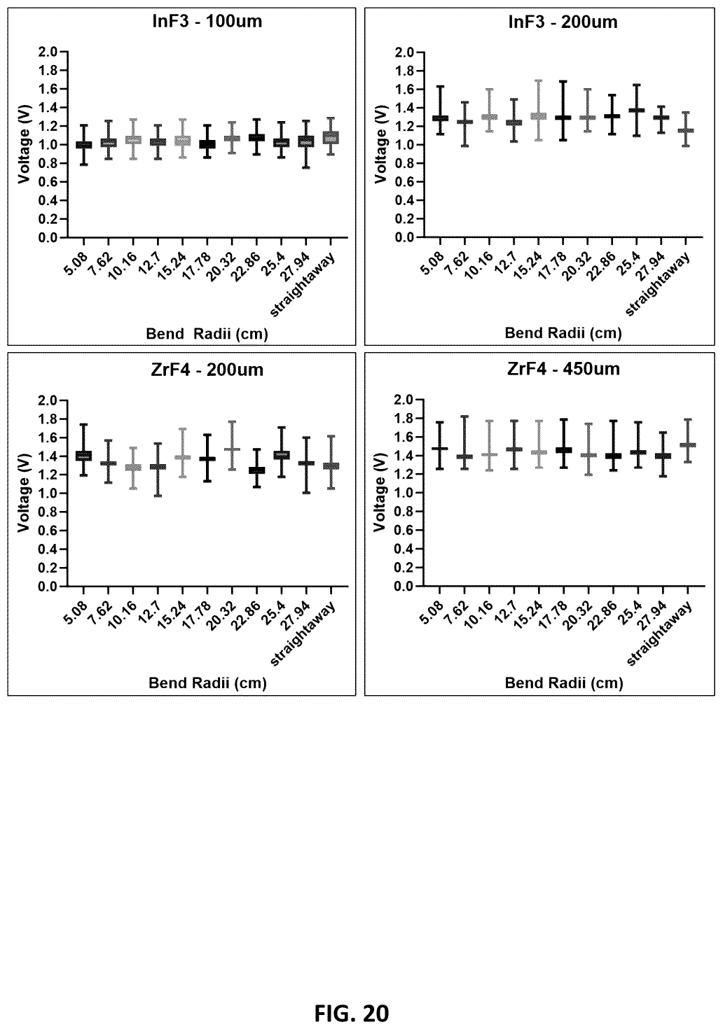

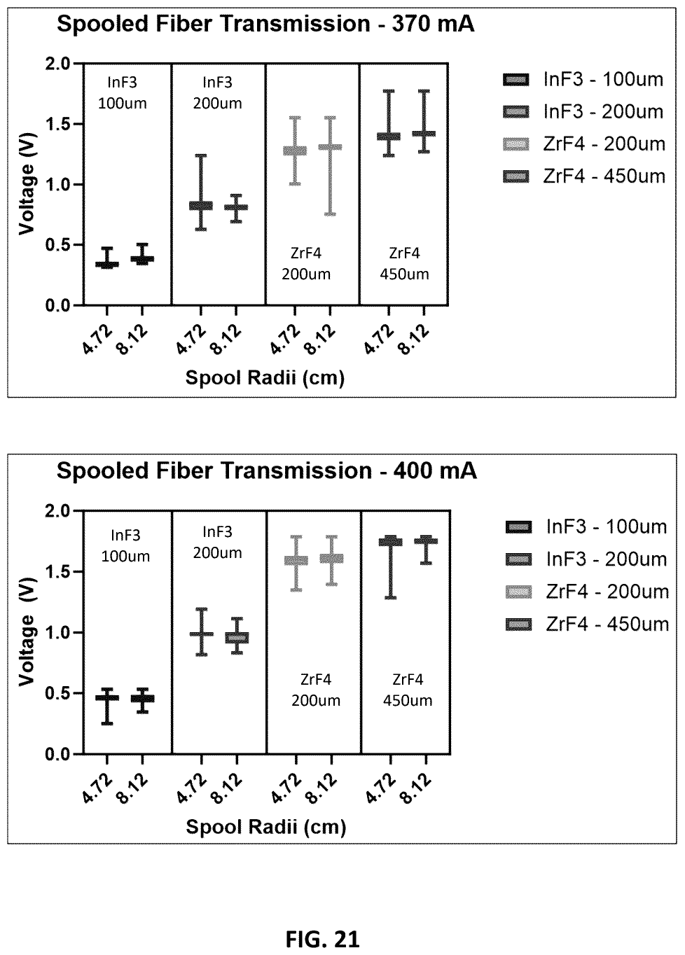

FIG. 17 illustrates a quantum cascade laser in a horizontal high heat load (HHLH) package. One example of such a laser is a QF4650HHLH-Fabry-Perot Quantum Cascade Laser. FIG. 18 further illustrates the fully assembled coupler.

When the fibers are used to therapeutically deliver energy to tissue within a body, the fibers may bend as the delivery needles may further have bend angles. This may result in bending loss.

It is contemplated that a system which provides for delivery of laser energy may account for any loss due to the coupling mechanism and due to the bending of fibers in order to provide more accurate dosage and delivery of laser energy. In order to evaluate bend loss from fibers additional studies were performed to both experimentally measure optical fiber transmission and perform computational simulations.

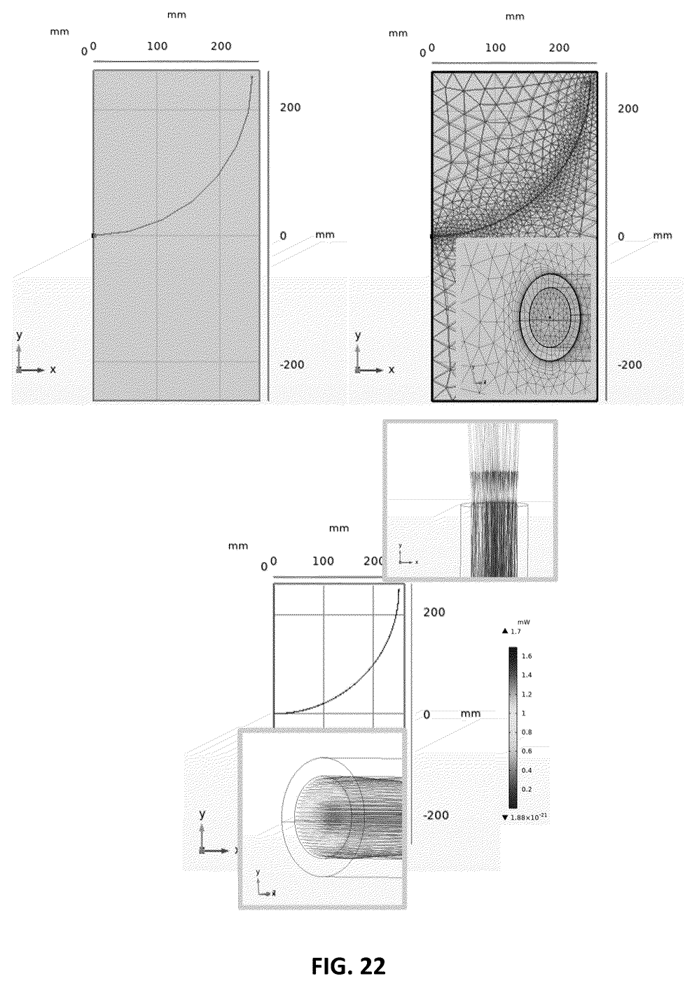

To experimentally measure the effect of bending on optical fiber transmission, experiments were designed to position fiber to exhibit different bend radii between a laser and a detector. The resulting voltage signal was from the detector was observed to determine the effect of the bend radii on the optical fiber transmission. In a first set of experiments, tracks defining varying bend radii were used to maintain the optical fiber in place. In another set of experiments, the optical fiber was wound around a spool from the laser to the detector. FIG. 19 panel A illustrates tracks configured such that the fiber maintains various bend radii. FIG. 19 panel B illustrates fiber wound around a spool from the laser to the detector. FIG. 19 panel C further illustrates fiber wound around the spool. FIG. 19 panel D illustrates a detector connected to a computer to display the detected voltage signal.

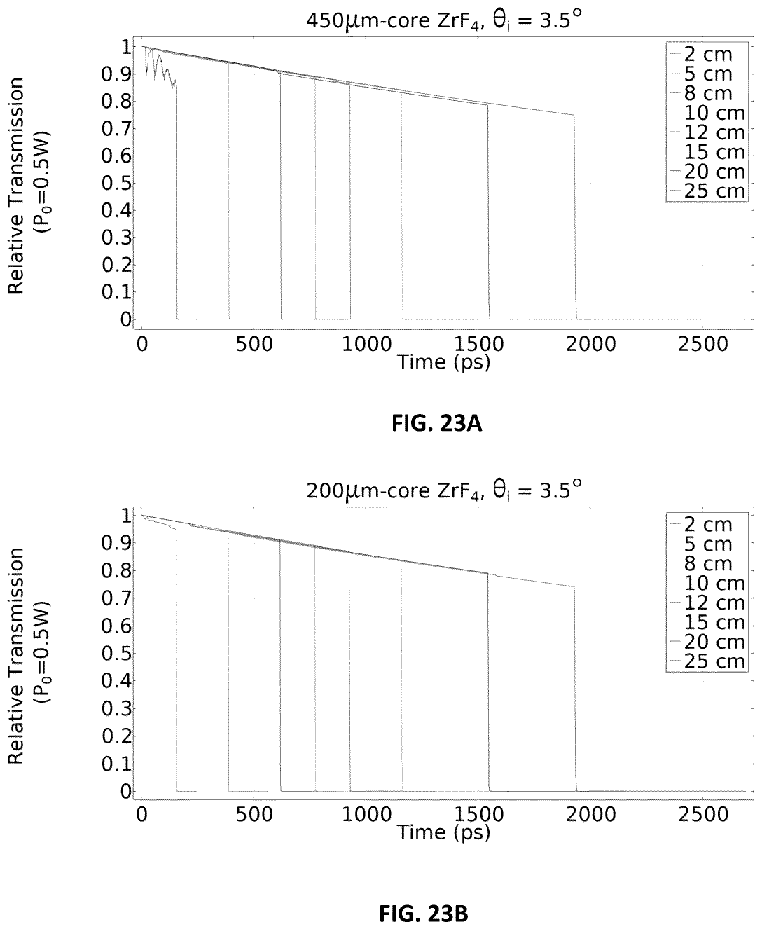

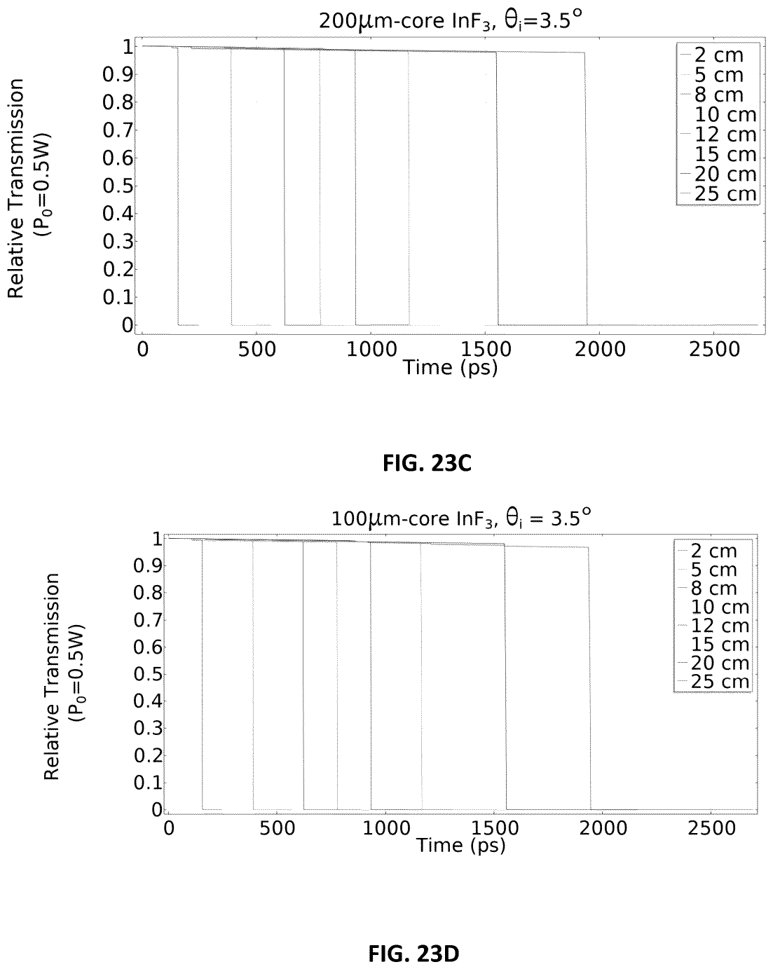

FIG. 20 illustrates results from the experimental testing for different types of fiber (InF3 and ZrF4) of different sizes where the bend radii was defined by the tracks. FIG. 21 illustrates results for different types of fibers and sizes.

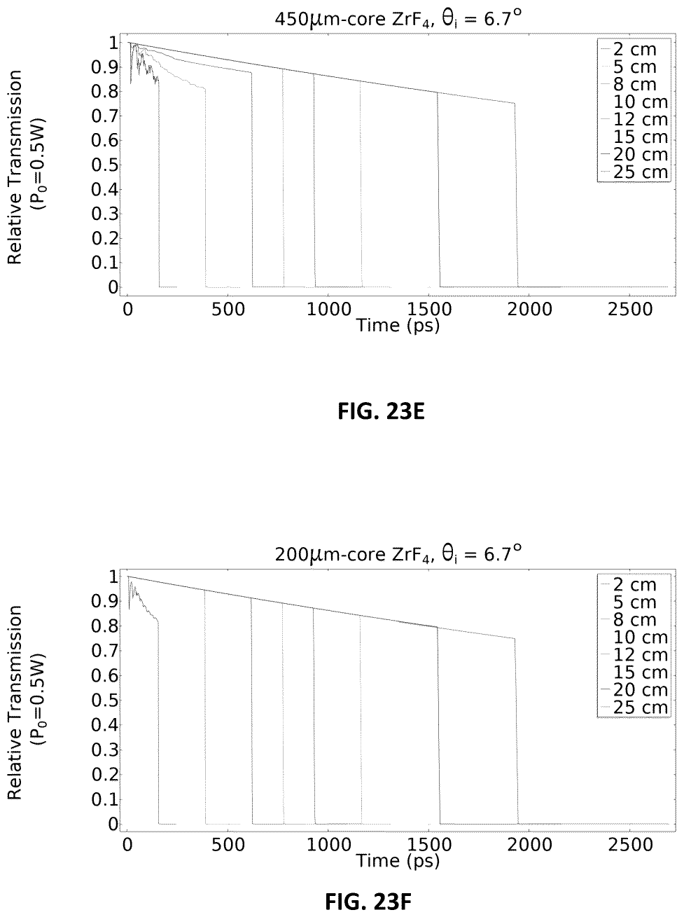

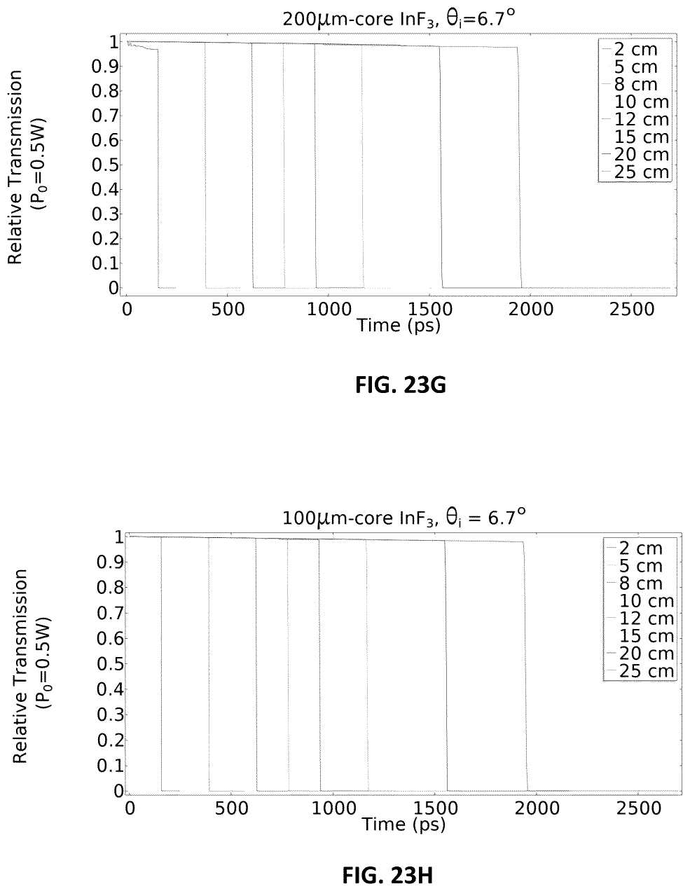

For the computational simulations, COMSOL was used to determine optical fiber bend loss. Geometry of the fiber was defined including the shape and size of the fiber and surrounding absorbing medium. Meshing was used to break up the geometry into small finite elements. Ray trajectories were used to compute how the rays move through the fiber. FIG. 22 illustrates representative geometry, meshing, and ray trajectories. FIGS. 23A, 23B, 23C, 23D, 23E, 23F, 23G, 23H illustrate results of the computational simulations for different angles, for different types of fibers, of different sizes.

Based on both the experimental measured and computationally simulated results, there are negligible bend loss in these optical fibers unless at sharp bends. This is a significant result in the context of the present disclosure as it is desirable that the delivery system allow for bending in order to improve positioning. Bending allows for better positioning of the laser to enhance laser ablation of tissue to be ablated while also better preserving tissue which does not require ablation. This enhanced precision may lead to improved patient outcomes. Moreover, where bending loss is negligible, less power is required to compensate for bending loss which may result in more compact design and/or reduced complexity of the system including in reducing complexity in the control of the laser output.

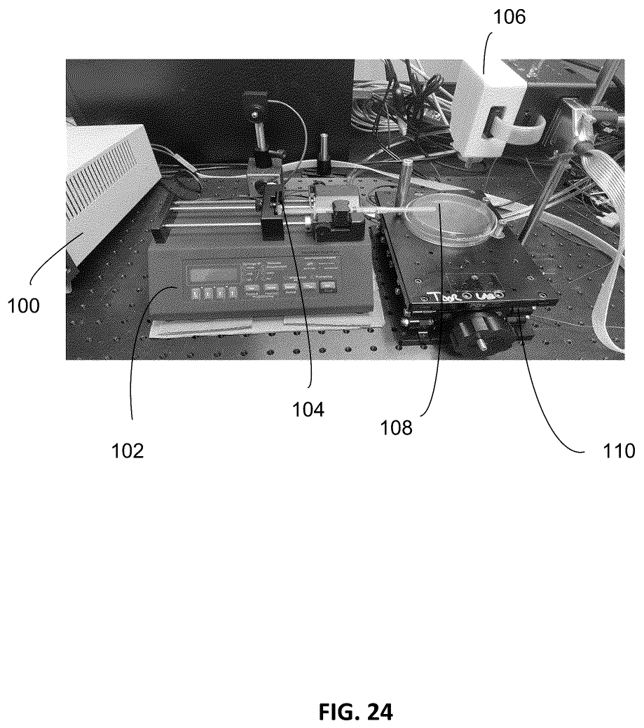

FIG. 24 illustrates an experimental setup which includes laser components and a programmable syringe pump. A laser driver and temperature controller 100 is shown along with a temperature controlled laser mount with fiber coupling port 106. A programmable syringe pump 102 is also shown. A single channel needle assembly 104 is shown to deliver fluid and laser energy to tissue which is present at the tissue holding platform 108 on a z-axis stage 110.

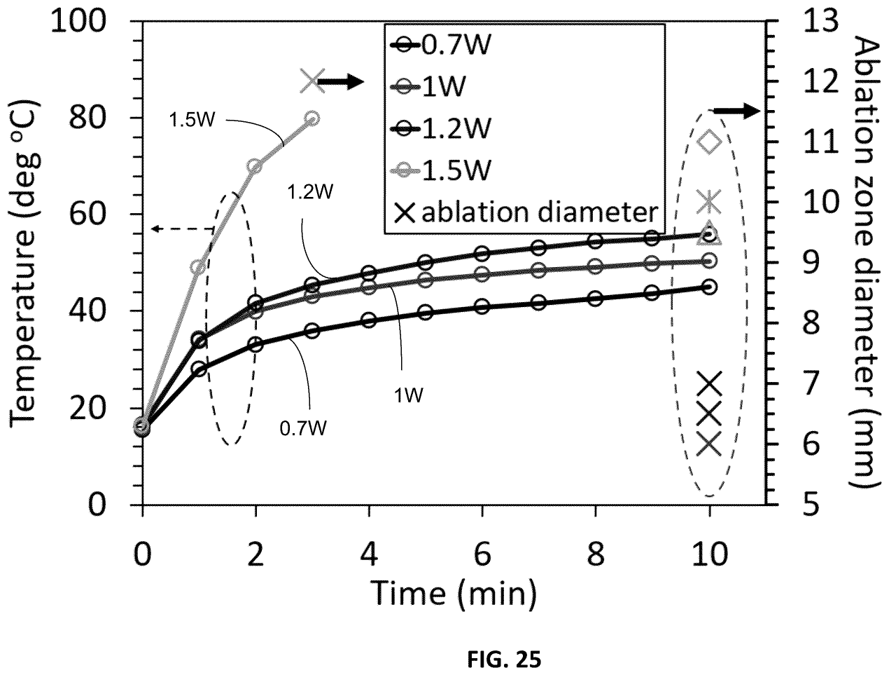

FIG. 25 is a figure illustrating results obtained using the experimental setup of FIG. 24 using lasers of different power output including 1.5 W, 1.2 W, 1 W, and 0.7 W. The time (minutes) and temperature (degrees Celsius) are shown as well as the ablation zone diameter.

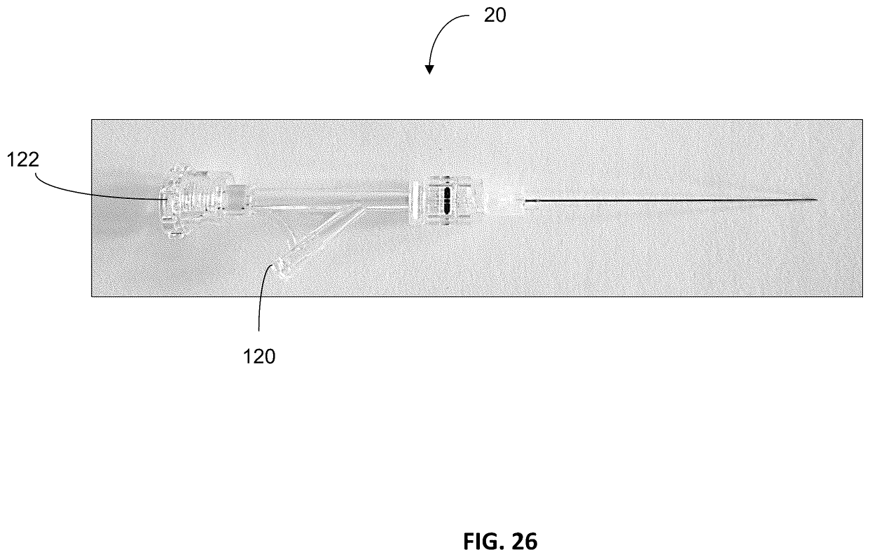

FIG. 26 illustrates an example of the needle assembly 20. The needle assembly 20 has a mid-IR fiber inlet 122 for receiving laser energy. The needle assembly 20 further has an inlet 120 for receiving fluid by injection such as saline or D5 W (5 percent dextrose in water).

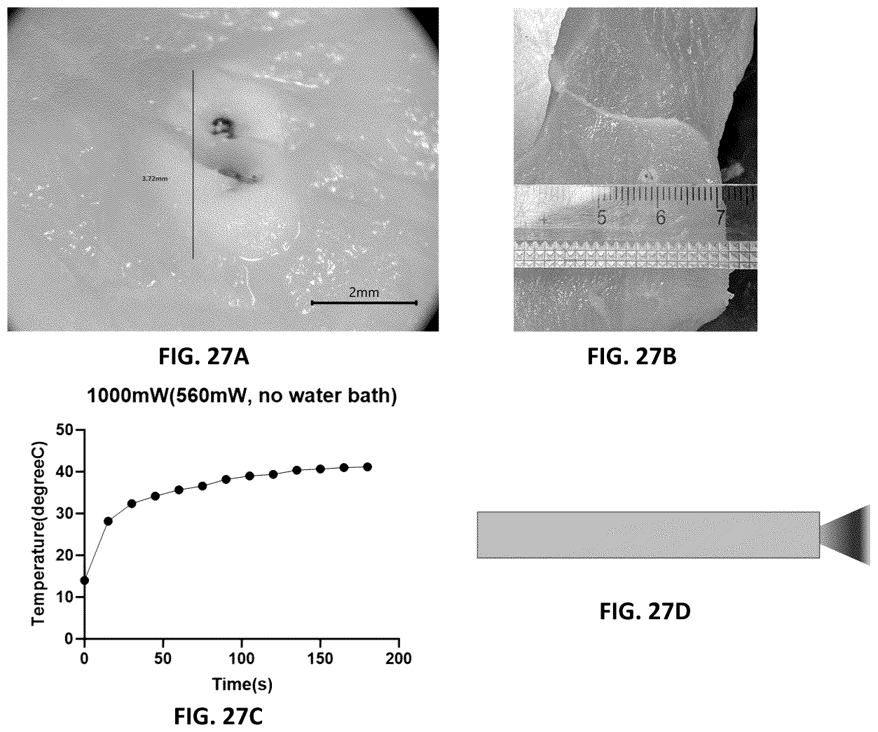

FIG. 27A is a photograph showing chicken breast tissue after fiber (untextured) ablation, FIG. 27B is a photograph showing the tissue, FIG. 27C is a graph showing temperature at different times using a 1000 mW/560 mW laser, no water bath. FIG. 27D illustrates that for untextured fiber, the mid-IR light comes out from the fiber end as confirmed by the tissue ablation.

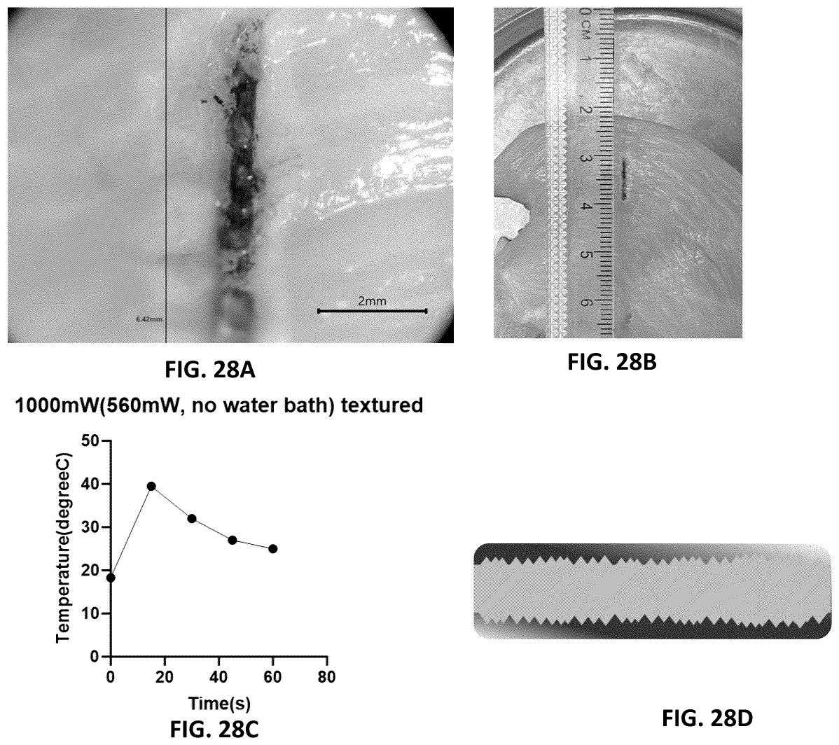

FIG. 28A is a photograph showing chicken breast tissue after fiber (textured) ablation with 1000 mW (560 mW) for 3 minutes, not water bath, placed in room temperature for 2 hours with the initial temperature 18.3 degrees. FIG. 28B is a further photograph showing the chicken breast. FIG. 28C is a graph showing temperatures at different times. The fiber was burnt in 25 seconds. FIG. 28D illustrates that with the textured fiber, the mid-IR light comes out longitudinally across the textured fiber length as confirm with the tissue ablation.

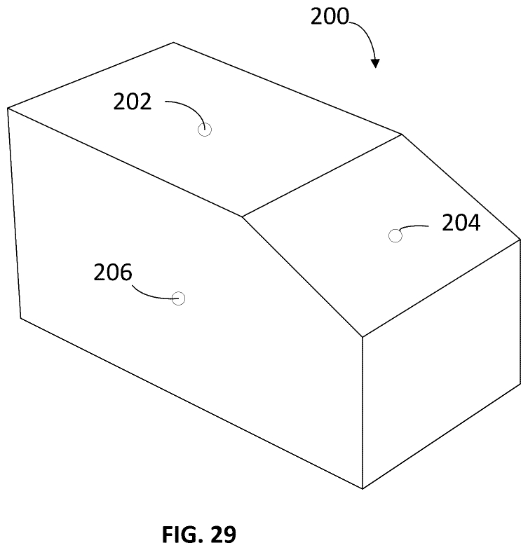

FIG. 29 illustrates one example for a laser interstitial thermal therapy probe which may be used for delivering laser energy. The design may be 3D printed or otherwise manufactured. The laser interstitial thermal therapy probe 200 may include a hole 202 for a fiber. A screw thread 206, and a hole 204 for the thermocouple.

It is contemplated that the multimodal laser energy and biochemical delivery system allow for greater control of therapeutic delivery such that tunable tissue ablation volumes may be achieved, for example, volumes of 5-100 cm³.

One example of a biochemical which may be used is talimogene laherparepvec (T-VEC) (commercial name: Imalygic 10), although any number of other therapeutic agents or radiopharmaceutical agents may be used. Moreover, the MIR laser radiation delivered by MIR-transparent fluoride fibers coupled to MIR lasers, threaded in the needles allows for high tunable and effective solid tumor ablation.

For example, it is contemplated that for a given tumor volume a MIR laser radiation dose (J/cm²) and radiance (mW/cm²) may be calculated to ablate the tumor volume. Such information may be communicated using a user interface of the device. In addition is contemplated that for a given tumor volume a MIR laser radiation dose and radiance as well as biochemical dosage parameters may also be calculated. It is contemplated that any number of different models may be used to provide such calculations, including machine learning models, AI models, biochemical dosage models (including without limitation, pharmacodynamic models and pharmacokinetic models), biochemical radiation dosimetry models, biophysical models, or other types of models. It is further contemplated that different laser exposure paradigms may be used such as continuous wave or pulsed exposure and any losses associated with coupling or bend of fibers may be taken into account.

Therefore, it is to be understood that various methods, systems, and apparatus have been shown and described herein. Different aspects are described both separately and in combination to provide a multimodal therapy delivery system. Although specific examples are provided, the present disclosure is not to be limited by or to the specific examples as numerous options, variations, and alternatives are contemplated. This includes variations in the needle assembly, variations in the wavelength, power, or other variations in the laser, variations in the coupling, variations in the type of fiber, variations in the texturing and method of texturing where the fibers are textured, variations in the user interface or display, variations in the control system used and control algorithms applied, variations in the delivery system, variations in the biochemical or therapeutic fluid used, variations in the number or types of fluids used, variations in the sensors used, and other variations.

1. A multimodal therapy delivery system configured to deliver both biochemical material and energy therapies, the multimodal therapy delivery system comprising:
at least one mid-infrared (MIR) laser;
at least one MIR transparent fluoride fiber coupled to each of the at least one MIR laser;
a needle assembly, with the at least one MIR transparent fluoride fiber threaded therethrough; and
wherein the multimodal therapy delivery system is configured to deliver biochemical material through the needle assembly and wherein the multimodal therapy delivery system is further configured to deliver energy from the at least one MIR laser through the at least one MIR transparent fluoride fiber threaded through the needle assembly.

2. The multimodal therapy delivery system of claim 1 wherein the needle assembly comprises a multi-prong needle for delivering the biochemical material.

3. The multimodal therapy delivery system of claim 1 wherein the needle assembly comprises a plurality of side holes positioned in a spiral fashion along a length of a needle within the needle assembly.

4. The multimodal therapy delivery system of claim 1 wherein the needle assembly comprises at least one curved channels and wherein the needle assembly comprises a side hole.

5. The multimodal therapy delivery system of claim 1 wherein the needle assembly further comprises an outer sheath.

6. The multimodal therapy delivery system of claim 1 further comprising a delivery mechanism configured to control flow of the biochemical material and delivery of light from the MIR laser.

7. The multimodal therapy delivery system of claim 6 wherein the delivery mechanism comprises a pump configured to separately delivery at least one biochemical material to a plurality of different channels of the needle assembly.

8. The multimodal therapy delivery system of claim 1 wherein the needle assembly comprises a metallic structure having a plurality of channels.

9. The multimodal therapy delivery system of claim 1 wherein each of the at least one MIR transparent fluoride fiber is coupled to a corresponding one of the at least one MIR laser with a coupling.

10. The multimodal therapy delivery system of claim 1 wherein each of the at least one MIR transparent fiber is textured for diffused light output.

11. The multimodal therapy delivery system of claim 1 further comprising a control system for operating the multimodal therapy delivery system wherein the control system provides for controlling amount of energy delivered from the at least one MIR laser to a target based on position of the at least one MIR transparent fluoride fiber and calculated losses associated with delivery of energy from the at least on MIR laser to the target.

12. The multimodal therapy delivery system of claim 1 wherein the at least one MIR transparent fluoride fiber comprises at least one of indium fluoride or zirconium fluoride.

13. The multimodal therapy delivery system of claim 1 further comprising:
at least one delivery mechanism operatively connected to one or more reservoirs of biochemical material;
a control system operatively connected to the at least one delivery mechanism and the at least one MIR laser; and
a display operatively connected to the control system.

14. A method comprising using the multimodal therapy delivery system of claim 1 to volumetrically ablate tissue by:
delivering at least one biochemical through a first channel of the needle assembly to tissue of a patient; and
simultaneously delivering energy from the at least one MIR laser to the tissue of the patient through the at least one MIR transparent fluoride fiber positioned within a second channel of the needle assembly.

15. The method of claim 14 wherein a volume of the tissue being volumetrically ablated is in a range of 5-100 cm³.

16. The method of claim 14 further comprising positioning the multimodal therapy delivery system by bending the at least one MIR transparent fluoride fiber prior to delivery of the energy from the at least one MIR laser.

17. The multimodal therapy delivery system of claim 1 wherein the needle assembly comprises:
a conduit needle having an outer sheath;
a plurality of channels in the conduit needle;
an exit hole from each of the plurality of channels;
at least one fluid injecting delivery needle for delivering a therapeutic fluid;
at least one energy delivery needle for delivering laser energy;
at least one laser fiber operatively connected to a corresponding one of the at least one energy delivery needle for threading through a corresponding one of the plurality of channels to deliver the laser energy; and
wherein the needle assembly is configured for delivering the therapeutic fluid and the laser energy simultaneously.

18. A therapy delivery system comprising:
at least one mid-infrared (MIR) laser having a wavelength of between about 3 and 13 microns;
a plurality of MIR transparent fibers coupled to one of the at least one MIR laser;
a coupling for each of the at least one of the plurality of MIR transparent fibers; and
wherein each of the plurality of MIR transparent fibers is textured.

19. The therapy delivery system of claim 18 wherein the therapy delivery system is further configured for delivering therapeutic fluid simultaneously with laser energy from the at least one MIR laser.

20. A method of volumetrically ablating a volume of tissue, the method comprising:
providing a multimodal therapy delivery system configured to deliver both biochemical material and energy therapies, the multimodal therapy delivery system including: (1) at least one mid-infrared (MIR) laser having a wavelength within a range of 3 to 13 μm; (2) at least one MIR transparent fluoride fiber coupled to each of the at least one MIR laser using a coupling; (3) a needle assembly, with the at least one MIR transparent fluoride fiber threaded therethrough; and (4) wherein the multimodal therapy delivery system is configured to deliver biochemical material through the needle assembly and wherein the multimodal therapy delivery system is further configured to deliver energy from the at least one MIR laser through the at least one MIR transparent fluoride fiber threaded through the needle assembly;
determining parameters at least based in part on the volume of the tissue being volumetrically ablated;
laser ablating the volume of tissue using the multimodal therapy delivery system; and
wherein the volume of the tissue being volumetrically ablated is in a range of 5-100 cm³.
